(19) United States
(12) Patent Application Publication
(10) Pub. No.: US 2009/0298934 A1
(43) Pub. Date: Dec. 3, 2009

(54) Diamidine Inhibitors of TDP1

(75) Inventors: Yves Pommier, Bethesda, MD (US); Christophe Marchand, Silver Sprig, MD (US)

Correspondence Address: EDWARDS ANGELL PALMER & DODGE LLP, PO BOX 55874, BOSTON, MA, 02205, US

(73) Assignee: GOVERNMENT OF THE US, AS REPRESENTED BY THE SECRET, Rockville, MD (US)

(21) Appl. No.: 12/225,672

(22) PCT Filed: Mar. 27, 2007
(86) PCT No.: PCT/US2007/007636

§ 371 Date: Jun. 22, 2009

Related U.S. Application Data
(60) Provisional application No. 60/786,604, filed on Mar. 27, 2006.

(51) Int. Cl. A61K 31/341; A61K 31/155; C07C 257/00; C07D 307/02

(52) U.S. Cl. 514/471; 514/631; 564/225; 549/491

(57) ABSTRACT

The instant invention is directed towards compounds, including diamidines, that inhibit Tdp1 and are useful in the treatment and/or prevention of cancer and parasitic disease.

### CROSS-REFERENCE TO RELATED APPLICATION

The present application claims the benefit of U.S. provisional application No. 60/786,604 filed Mar. 27, 2006, which is incorporated by reference herein in its entirety.

### STATEMENT OF GOVERNMENTAL INTEREST

This invention was funded by the National Cancer Institute at the National Institutes of Health. The United States Government has certain rights in this invention.

### BACKGROUND

Cancer, in all its manifestations, remains a devastating disorder. Although cancer is commonly considered to be a single disease, it actually comprises a family of diseases wherein normal cell differentiation is modified so that it becomes abnormal and uncontrolled. As a result, these malignant cells rapidly proliferate. Eventually, the cells spread or metastasize from their origin and colonize other organs, eventually killing their host. Due to the wide variety of cancers presently observed, numerous strategies have been developed to destroy cancer within the body.

Typically, cancer is treated by chemotherapy, in which highly toxic chemicals are given to the patient, or by radiotherapy, in which toxic doses of radiation are directed at the patient. Unfortunately, these “cytotoxic” treatments also kill extraordinary numbers of healthy cells, causing the patient to experience acute debilitating symptoms including nausea, diarrhea, hypersensitivity to light, hair loss, etc. The side effects of these cytotoxic compounds limits the frequency and dosage at which they can be administered. Such disabling side effects can be mitigated to some degree by using compounds that selectively target cycling cells, i.e., interfering with DNA replication or other growth processes in cells that are actively reproducing. Since cancer cells are characterized by their extraordinary ability to proliferate, such protocols preferentially kill a larger proportion of cancer cells in comparison to healthy cells, but cytotoxicity and ancillary sickness remains a problem.

Another strategy for controlling cancer involves the use of signal transduction pathways in malignant cells to “turn off” their uncontrolled proliferation, or alternatively, instruct such cells to undergo apoptosis. Such methods of treating cancer are promising but a substantial amount of research is needed in order to make these methods viable alternatives.

The treatment and/or cure of cancer has been intensely investigated culminating in a wide range of therapies. Cancer has been typically treated with surgery, radiation and chemotherapy, alone or in conjunction with various therapies employing drugs, biologic agents, antibodies, and radioactive immunoconjugates, among others. The common goal of cancer treatment has been, and continues to be, the elimination or amelioration of cancerous tumors and cells with minimal unpleasant or life-threatening side effects, due to toxicity to normal tissues and cells. However, despite efforts, these goals remain largely unmet.

In view of the above considerations, it is clear that there is a need to supplement existing methods of inhibiting cancer cell invasiveness and metastasis. Current approaches to cancer treatment frequently rely on highly cytotoxic compounds that cause ancillary debilitating sickness in patients, or use methodology that is expensive, procedurally difficult, and unpredictable.

### BRIEF SUMMARY OF THE INVENTION

Accordingly, it is one of the purposes of this invention to overcome the above limitations in cancer treatment, by providing compounds and methods for inhibiting the growth processes characteristic of cancer cells, including inhibiting invasiveness and metastasis, as well as inducing regression of primary tumors. In particular, it is desirable to identify anticancer compounds and methods that inhibit cancer growth specifically and with relatively high activity, i.e., being active at doses that are substantially free of harmful side effects. Additionally, it is a purpose of the invention to provide methods and compositions suitable for the development, identification, and/or characterization of compounds that are capable of modulating the activity of tyrosyl-DNA phosphodiesterases (TDPs), particularly tyrosyl-DNA phosphodiesterase 1 (TDP1). The present invention provides means to identify and characterize compounds that are suitable for inhibiting TDP activity in vivo and in vitro.

Thus, in one aspect, the invention provides a method of inhibiting Tdp1 activity in a subject. The method includes the step of administering to the subject a diamidine compound capable of modulating the activity of Tdp1.

In preferred embodiments, the diamidine compound is a compound of Formula I:

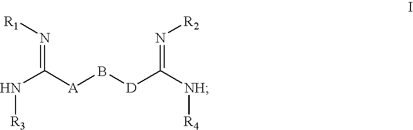

in which A, B and D are each independently C₁-C₆ alkylene, C₃-C₁₀ cycloalkylene, C₁-C₉ heterocycloalkylene, C₆-C₁₀ arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S((O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, akynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In further preferred embodiments, A and D are each C₆-C₁₀ arylene and B is heteroarylene; more preferably, B is furanylene. In a preferred embodiment, the compound is one of the following (i.e. 2,5-di-(4-phenylamidine)furan and 2,5-di-(4-phenylamidine)-3,4-dimethylfuran) or pharmaceutically acceptable salts thereof:

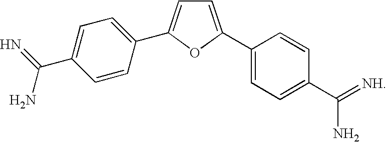

### 2,5-di-(4-phenylamidine)furan

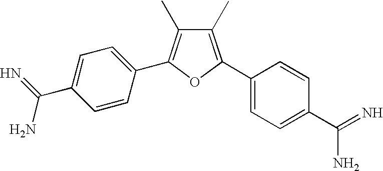

### 2,5-di-(4-phenylamidine)-3,4-dimethylfuran

In another aspect, the invention provides a method of inhibiting Tdp1 activity in a subject identified as being in need of such treatment. The method includes the step of administering to the subject a diamidine compound, wherein the diamidine compound is capable of binding to Tdp1.

In another aspect, the invention provides a method treating a Tdp1-related disorder in a subject. The method includes the step of administering to the subject an effective amount of a diamidine compound, such that the subject is treated for the disorder, and the disorder is cancer, tumor, neoplasm, neovascularization, vascularization, cardiovascular disease, intravasation, extravasation, metastasis, arthritis, infection, Alzheimer's Disease, blood clot, atherosclerosis, melanoma, skin disorder, rheumatoid arthritis, diabetic retinopathy, macular edema, or macular degeneration, inflammatory and arthritic disease, or osteosarcoma.

In another aspect, the invention provides a method of treating cancer in a subject identified as in need of such treatment. The method includes the step of administering to the subject an effective amount of a compound of Formula I:

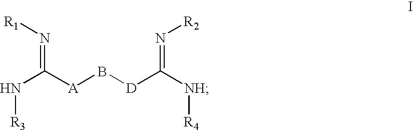

in which A, B and D are each independently C₁-C₆ alkylene, C₃-C₁₀ cycloalkylene, C₁-C₉ heterocycloalkylene, C₆-C₁₀ arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S((O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, akynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In further preferred embodiments, A and D are each C₆-C₁₀ arylene and B is heteroarylene; more preferably, B is furanylene. In a preferred embodiment, the compound one of the following or a pharmaceutically acceptable salt thereof:

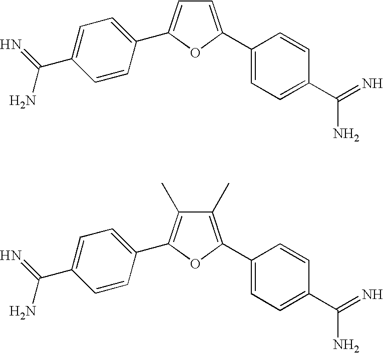

In further preferred embodiments, the compound is a Tdp1 inhibitor. In further preferred embodiments, the method further includes an additional therapeutic agent; preferably the additional therapeutic agent is an anticancer compound, more preferably a TopI inhibitor.

In further preferred embodiments, the step of administering the compound includes administering the compound orally, topically, parentally, intravenously or intramuscularly. In further preferred embodiments, the method includes the step of administering an effective amount of a composition including a diamidine compound and a pharmaceutically suitable excipient. In further preferred embodiments, the subject is a human.

In another aspect, the invention provides a pharmaceutical composition. The pharmaceutical composition includes a compound of Formula I (above) in which A, B and D are each independently C₁-C₆ alkylene, C₃-C₁₀ cycloalkylene, C₁-C₉ heterocycloalkylene, C₆-C₁₀arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted; or a pharmaceutically-acceptable salt thereof; together with a pharmaceutically-acceptable carrier or excipient.

In another aspect, the invention provides a compound of Formula I:

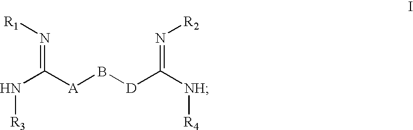

in which A, B and D are each independently C₁-C₆ alkylene, C₃-C₁₀cycloalkylene, C₁-C₉ heterocycloalkylene, C₆-C₁₀ arylene, C₁-C₁₀heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In certain preferred embodiments, the compound is identified by a method for identifying a compound which modulates the activity of a Tyrosyl-DNA phosphodiesterase (Tdp1).

In another aspect, the invention provides the use of a compound in the manufacture of a medicament for inhibiting or reducing cancer in a patient, the compound being of Formula I:

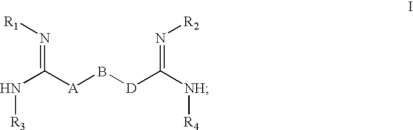

in which A, B and D are each independently C₁-C₆ alkylene, C₃-C₁₀ cycloalkylene, C₁-C₉ heterocycloalkylene, C₆-C₁₀arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In another aspect, the invention provides a kit. The kit includes an effective amount of a diamidine compound according to the invention in unit dosage form, together with instructions for administering the compound to a subject suffering from cancer.

In still another aspect, the invention provides a method for identifying a compound that modulates the interaction of Tdp1 with a Tdp1 substrate. The method includes the steps of obtaining a crystal structure of Tdp1 or obtaining information relating to the crystal structure of Tdp1, in the presence and/or absence of a Tdp1 substrate, and modeling a test compound into or on the substrate binding site of the crystal structure to determine whether the compound modulates the interaction of Tdp1 with a substrate.

### BRIEF DESCRIPTION OF THE DRAWINGS

FIGS. 1A-1D show Compounds (a) and (b) (see Example 1) (1RFF) (shown in ball-and stick) docked in the binding site of the Tdp1 N domain.

FIGS. 2A-2D show Compounds (a) and (b) (1NOP) (shown in ball-and stick) docked in the binding site of the Tdp1 N domain.

FIGS. 3A-3D show Compounds (a) and (b) (1RHO) (shown in ball-and stick) docked in the binding site of the Tdp1 N domain.

FIG. 4 is a table showing the structures and the activity of certain amidine and diamidine compounds against Tdp1.

FIG. 5. High-throughput electrochemiluminescene assay developed to identify novel Tdp1 inhibitors. A, Coupling reaction to generate the electrochemniluminescent (ECL) substrate (BV-14Y). The ruthenium-containing tag (NHS ester BV-Tag; from BioVeris Corp.) is coupled to the DNA substrate [14Y (sequence as in 3A) linked to a biotin at its 5′ end]. After coupling, the BV tag is attached to the phosphotyrosine of the 14Y DNA forming the BV-14Y DNA after the release of a succinimide group. The labeled material is then purified on an oligo spin column. B, Tdp1 catalytic reaction leading to the processing of the Tdp1-BV-Tag DNA substrate. Tdp1 cleaves the phosphotyrosine removing the tyrosine-BV-Tag group and leaving a 3′ phosphate on the DNA. This leads to a loss of the chemiluminescence signal. Positive hits for potential Tdp1 inhibitors prevent this loss of signal. C, Signal response curve in the presence of increasing concentrations (nM) of Tdp1. The ECL signal is lost when the Tdp1 concentration is increased.

FIG. 6. Identification of 2,5-di-(4-phenylamidine)furanas a Tdp1 inhibitor by high-throughput electrochemiluminescene assay. A, Graph representing the effect of 1981 compounds in the NCI-DTP diversity set on Tdp1 activity at 10 pM. Each dot indicates a signal value for a tested sample. The substrate chemiluminescence (Arbitrary units; A.U.) in the absence of Tdp1 averages at 16313±1084 (n=200; where “n” indicates the number of samples). In the presence of Tdp1 the loss of signal averages at 8784±559 (n=100). The effect of 1981 compounds screened is represented. Positive Tdp1 inhibitors prevent the loss of signal. Dashed line represents 50% inhibition of Tdp1. 2,5-di-(4-phenylamidine)furan gives a signal value of 16910 (indicated by an arrow) which corresponds to 100% Tdp1 inhibition at 10 μM. B, Table showing the effect of 10 μM 2,5-di-(4-phenylamidine)furan on Tdp1 activity as measured by the restoration of the electrochemiluminiscent signal. Vanadate at 10 mM was used for comparison.

FIG. 7. Inhibition of Tdp1 activity by 2,5-di-(4-phenylamidine)furan. A, Schematic representation of the Tdp1 biochemical assay. The partially duplex oligopeptide D14Y or single stranded 14Y was used as a substrate. ³²P-Radiolabeling (*) was at the 5′ terminus of the 14-mer strand. Tdp1 catalyzes the hydrolysis of the 3′-phosphotyrosine bond and converts 14Y and D14Y to an oligonucleotide with 3′-phosphate, 14P or D14P respectively. B, gel showing Tdp1 inhibition by 2,5-di-(4-phenylamidine)furan in both single-strand (14Y) and partially duplex (D14Y) substrates. Reactions were performed at pH 8.0 with 25 nM 14Y or D14Y, 1 ng of Tdp1, and the indicated concentrations (μM) of 2,5-di-(4-phenylamidine)furanat 25° C. for 20 min. Arrows indicate the 3′-phosphate oligonucleotide product (14P) that runs quicker than the corresponding tyrosyl oligonucleotide substrate (14Y) in a denaturing PAGE. The duplex D14Y substrate and D14P product are detected on the gel by their corresponding labeled single strands (14Y and 14P), as they are no longer annealed under the denatured conditions. C, densitometry analysis of the gel shown in panel B. Tdp1 activity was calculated as the percentage of 14Y converted to 14P as a function of the concentration of 2,5-di-(4-phenylamidine)furan. The horizontal line corresponds to 50% inhibition of Tdp1 activity.

FIG. 8. Binding of 2,5-di-(4-phenylamidine)furan (25 mM-97 mM) to a 495 RU surface of a stem-loop oligonucleotide (A) and 504 RU surface of a single-stranded oligonucleotide (13). The equilibrium level of binding was determined for each 2,5-di-4-phenylamidine)furan concentration for the stem-loop oligonucleotide (C) or the single-stranded oligonucleotide (D). The graphs represent a fit using a 2 binding site model for the stem-loop oligonucleotide (C) or a single binding site model for the singlestranded oligonucleotide (D).

FIG. 9. Kinetics of Tdp1 inhibition by 2,5-di-(4-phenylamidine)furan. A, a 100-μ reaction mixture containing 25 nM 14Y and 5 ng of Tdp1 was incubated at pH 8.0 at 25° C. in the absence of drug, or in the presence of 30, 60 or 120 μM 2,5-di-(4-phenylamidine)furan. Aliquots were taken at the indicated times (min). Reaction products were analyzed by denaturing PAGE. B, densitometry analysis of the gel shown in A. Tdp1 activity measured as the percentage of DNA substrate 14Y converted to 14P (Left panel) or substrate 14Y remaining (Right panel) as a function of reaction time. C, Reactions (20 μl) containing 25 nM 14Y and indicated amounts (ng) of Tdp1 were carried out in the absence or presence of 30, 60 or 250 μM 2,5-di-(4-phenylamidine)furan at 25° C., pH 8, for 20 min. A representative gel is shown. D, densitometry analysis of the gel shown in C. Tdp1 activity was calculated as the percentage of DNA substrate 14Y converted to 14P. The vertical line corresponds to 50% inhibition of Tdp1 activity.

FIG. 10. Inhibition of Tdp1 by 2,5-di-(4-phenylamidine)furanis independent of the DNA sequence. A, Sequences of the oligonucletotide substrates 14Y and 14Y-CC, which differ in their 3′-terminal bases being a -TT or a -CC that is linked to the phosphotyrosine. B, Reactions (100 μl) containing either 25 nM 14Y or 14Y-CC and 5 ng of Tdp1 was incubated at pH 8.0 at 25° C. Aliquots were taken at the indicated times (min). Reaction products were analyzed by denaturing PAGE. C, densitometry analysis of the gel shown in B. Tdp1 activity measured as the percentage of DNA substrates 14Y or 14Y-CC converted to their corresponding products as a function of reaction time. D, Reactions (20 μl) containing 25 nM 14Y or 14Y-CC and 1 ng Tdp1 were carried out in the presence of indicated concentrations (μM) of 2,5-di-(4-phenylamidine)furan at 25° C., pH 8, for 20 min. A representative gel is shown. E, densitometry analysis of the gel shown in D. Tdp1 activity was calculated as the percentage of DNA substrates 14Y or 14Y-CC converted to their product. The horizontal line corresponds to 50% inhibition of Tdp1 activity.

FIG. 11. Structure-activity of 2,5-di-(4-phenylamidine)furan, Berenil and Pentamidine. A, Comparison of the chemical structures of 2,5-di-(4-phenylamidine)furan, Berenil and Pentamidine. R; common chemical moiety. B, Reactions were performed with indicated concentrations (pM) of 2,5-di-(4-phenylamidine)furan, Berenil and Pentamidine for 20 min at pH 8.0 and 25° C. in the presence of 25 nM 14Y substrate and 1 ng of Tdp1. Samples were separated on a 20% Urea-PAGE gel and visualized.

### DETAILED DESCRIPTION

### Definitions

In order that the invention may be more readily understood, certain terms are first defined and collected here for convenience.

The term “administration” or “administering” includes routes of introducing the compound(s) to a subject to perform their intended function. Examples of routes of administration which can be used include injection (subcutaneous, intravenous, parenterally, intraperitoneally, intrathecal), oral, inhalation, rectal and transdermal.

The term “admixture” refers to something that is produced from mixing.

The term “alkyl” refers to the radical of saturated aliphatic groups, including straight-chain alkyl groups and branched-chain alkyl groups. The term alkyl further includes alkyl groups, which can further include oxygen, nitrogen, sulfur or phosphorous atoms replacing one or more carbons of the hydrocarbon backbone. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Most preferred are lower alkyls.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Preferably a lower alkyl has no heteroatoms in its backbone structure.

The terms “alkylaryl” or “aralkyl” are used interchangeably, and refer to an alkyl substituted with an aryl (e.g., phenylmethyl(benzyl)), or an aryl group substituted with an alkyl. The term “heteroaralkyl” refers to either an alkylaryl or aralkyl groups that is substituted at any number of positions with a heteroatom.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively.

The term “aryl” as used herein, refers to the radical of aryl groups, including 5- and 6-membered single-ring aromatic groups. “Heteroaryl” groups may include from one to four heteroatoms. Examples of aryl and heteroaryl groups include benzene, pyrrole, furan, thiophene, imidazole, benzoxazole, benzothiazole, triazole, tetrazole, pyrazole, pyridine, pyrazine, pyridazine and pyrimidine, and the like. Polycyclic fused aromatic groups such as naphthyl, quinolyl, indolyl, and the like are also contemplated.

Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” includes all genomic and non-genomic activities elicited by these compounds.

The term “cancer” refers to a malignant tumor of potentially unlimited growth that expands locally by invasion and systemically by metastasis. The term “cancer” also refers to the uncontrolled growth of abnormal cells. Specific cancers are selected from, but not limited to, rhabdomyosarcomas, chorio carcinomas, glioblastoma multiformas (brain tumors), bowel and gastric carcinomas, leukemias, ovarian cancers, prostate cancers, lymphomas, osteosarcomas or cancers which have metastasized.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “chiral” refers to molecules which have the property of non-superimposability of the mirror image partner, while the term “achiral” refers to molecules which are superimposable on their mirror image partner.

The term “cycloalkyl” refers to the radical of saturated or unsaturated cyclic aliphatic groups, including cycloalkyl (alicyclic) groups, alkyl substituted cycloalkyl groups, and cycloalkyl substituted alkyl groups. The term cycloalkyl further includes cycloalkyl groups, which can further include oxygen, nitrogen, sulfur or phosphorous atoms replacing one or more carbons of the hydrocarbon backbone. Preferred cycloalkyls have from 3-10 carbon atoms in their ring structure, and more preferably have 3, 4, 5, 6 or 7 carbons in the ring structure. Preferably a cycloalkyl has no heteroatoms in its ring structure.

The term “diamidine compound” as used herein, refers to a compound having two or more amidine groups, including unsubstituted amidine (—C(NH)NH₂) groups and substituted amidine groups (—C(NR₁)NHR₂) in which R₁ and R₂ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted. Compounds having both unsubstituted and substituted amidine(s) are also included. In certain embodiments, unsubstituted amidine groups are preferred.

The term “diastereomers” refers to stereoisomers with two or more centers of dissymmetry and whose molecules are not mirror images of one another.

The term “deuteroalkyl” refers to alkyl groups in which one or more of the of the hydrogens has been replaced with deuterium.

DNA molecules are said to have “5′ ends” and “3′ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the angiogenesis inhibitor compound are outweighed by the therapeutically beneficial effects.

A therapeutically effective amount of compound (i.e., an effective dosage) may range from about 0.001 μg/kg/day to 500 mg/kg/day of body weight, preferably about 1 μg/kg/day to 100 mg/kg/day, still more preferably about 10 μg/kg/day to 50 mg/kg/day body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. It will also be appreciated that the effective dosage of a compound used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

The term “heterocycloalkyl” refers to the radical of saturated or unsaturated cyclic aliphatic groups substituted by any number of heteroatoms, including heterocycloalkyl (alicyclic) groups, alkyl substituted heterocycloalkyl groups, and heterocycloalkyl substituted alkyl groups. Heteroatoms include but are not limited to oxygen, nitrogen, sulfur or phosphorous atoms replacing one or more carbons of the hydrocarbon backbone. Preferred heterocycloalkyls have from 3-10 carbon atoms in their ring structure, and more preferably have 3, 4, 5, 6 or 7 carbons in the ring structure, wherein a heteroatom may replace a carbon atom.

The terms “hyperproliferative” and “neoplastic” are used interchangeably, and include those cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “inhibition” and “inhibits” refer to a method of prohibiting a specific action or function.

The term “inhibitor,” as used herein, refer to a molecule, compound or complex which blocks or modulates a biological or immunological activity.

The term “isomers” or “stereoisomers” refers to compounds which have identical chemical constitution, but differ with regard to the arrangement of the atoms or groups in space:

The term “leukemia” is intended to have its clinical meaning, namely, a neoplastic disease in which white corpuscle maturation is arrested at a primitive stage of cell development. The condition may be either acute or chronic. Leukemias are further typically categorized as being either lymphocytic i.e., being characterized by cells which have properties in common with normal lymphocytes, or myelocytic (or myelogenous), i.e., characterized by cells having some characteristics of normal granulocytic cells. Acute lymphocytic leukemia (“ALL”) arises in lymphoid tissue, and ordinarily first manifests its presence in bone marrow. Acute myelocytic leukemia (“AML”) arises from bone marrow hematopoietic stem cells or their progeny. The term acute myelocytic leukemia subsumes several subtypes of leukemia: myeloblastic leukemia, promyelocytic leukemia, and myelomonocytic leukemia. In addition, leukemias with erythroid or megakaryocytic properties are considered myelogenous leukemias as well.

The term “leukemic cancer” refers to all cancers or neoplasias of the hemopoietic and immune systems (blood and lymphatic system). Chronic myelogenous leukemia (CML), also known as chronic granulocytic leukemia (CGL), is a neoplastic disorder of the hematopoietic stem cell.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result In preferred embodiments, this phrase is intended to include hyperactive conditions that result in pathological disorders.

The term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangably, as their context will reveal, referring to generally to cells experiencing abnormal cell growth rates. Neoplasias and hyperplasias include “tumors,” which may be either benign, premalignant or malignant.

The term “non-direct interaction” refers to any interactions that are not ionic nor covalent, such as hydrogen bonding or van der Waals interactions.

The term “optionally substituted” can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted as a substituent can themselves be substituted, if appropriate.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intasternal injection and infusion.

A “peptide” is a sequence of at least two amino acids. Peptides can consist of short as well as long amino acid sequences, including proteins.

The terms “polycyclic group” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g. benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The term “protein” refers to series of amino acid residues connected one to the other by peptide bonds between the alpha-amino and carboxy groups of adjacent residues. In general, the term “protein” is used to designate a series of greater than 50 amino acid residues connected one to the other.

The language “reduced toxicity” is intended to include a reduction in any undesired side effect elicited by a compound when administered in vivo.

The term “sarcoma” is art recognized and refers to malignant tumors of mesenchymal derivation.

The term “subject” refers to animals such as mammals, including, but not limited to, primates (e.g., humans), cows, sheep, goats, horses, dogs, cats, rabbits, rats, mice and the like. In certain embodiments, the subject is a human.

The term “sulfhydryl” or “thiol” means —SH.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly modulate the activity of Tdp1.

The term “therapeutically effective amount” refers to that amount of the compound being administered sufficient to prevent development of or alleviate to some extent one or more of the symptoms of the condition or disorder being treated.

The terms “treating” and “treatment” refer to a method of alleviating or abating a disease and/or its attendant symptoms.

The term “tumor suppressor gene” refers to a gene that acts to suppress the uncontrolled growth of a cancer, such as a tumor.

As used herein, the terms “tyrosine-DNA phosphodiesterase” and “TDP” refer to a protein that is encoded by a tyrosine-DNA phosphodiesterase gene sequence or to a protein. In addition, the terms refer to enzymes that cleave the phosphodiester bond linking the active site tyrosine residue of topoisomerase I with 3′-terminus of DNA in topo I-DNA complexes.

The indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

### Assays of the Invention

The present invention describes an assay for potential drugs or agents which modulate Tdp1 activity. Tdp1 repairs Top1-DNA covalent complexes by hydrolyzing the tyrosyl-DNA bond. Top1 relieves DNA torsional stress and relaxes DNA supercoiling by introducing DNA single-strad breaks. Top1 is the target of the anticancer agent camptothecin. Top1 inhibitors damage DNA by trapping covalent complexes between the Top1 catalytic tyrosine and the 3′ end of the broken DNA. Therefore, the drug or agent acts as a therapeutic for modulating tumor growth and metastasis.

There are a variety of assay formats that can be used to screen for modulators of Tdp1 activity. For a general description of different formats for binding assays, see Basic and Clinical Immunology, 7th Ed. (D. Stiles and A. Terr, ed.)(1991); Enzyme Immunoassay, E. T. Maggio, ed., CRC Press, Boca Raton, Fla. (1980); and “Practice and Theory of Enzyme Immunoassays” in P. Tijssen, Laboratory Techniques in Biochemistry and Molecular Biology, Elsevier Science Publishers, B.V. Amsterdam (1985), each of which is incorporated by reference.

Measurements of Tdp1 activity can be performed using a variety of assays. For example, the effects of the inventive compounds upon cancer can be measured by examining parameters described above. A suitable physiological change that affects activity can be used to assess the influence of a Tdp1-compound complex on the tag of the Tdp1 substrate. When the functional consequences are determined using intact cells or animals, one can also measure a variety of effects such as, tumors, tumor growth, tumor metastasis, neovascularization, hormone release, transcriptional changes to both known and uncharacterized genetic markers (e.g., northern blots), changes in cell metabolism such as cell growth or pH changes, and changes in intracellular second messengers such as cGMP.

Assays to identify compounds with modulating activity can be performed in vitro. For example, one assay for screening compounds for Tdp1 activity has been reported (see, e.g., M. C. Rideout et al., Nucleic Acids Res. 2004; 32(15): 4657-4664)

Moreover, once initial candidate compounds are identified, variants can be further screened to better evaluate structure activity relationships.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g. albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

An assay of the invention generally comprises contacting Tdp1 with the test compound to form a Tdp1-compound complex. Optionally, such contact can occur in solution, e.g., TRIS buffer, or phosphate buffered saline (PBS) at physiological pH.

As referred to herein, a compound (such as a diamidine compound) is capable of modulating (such as inhibiting) the activity of Tpd1 as may be assessed by the in vitro assay of Example 4 which follows and shows increased modulating activity relative to control (e.g. no sample or compound known not to modulate Tpd1 activity).

In certain embodiments of an assay of the invention, high throughput screening methods involve providing a library containing a large number of potential therapeutic compounds (candidate compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks.

Preparation and screening of combinatorial chemical libraries are understood by those of ordinary skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random biooligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 69096913), vinylogous polypeptides (Hagihara et al. (1992) J. Amer. Chem. Soc. 114: 6568), nonpeptidal peptidomimetics with a Beta D Glucose scaffolding (Hirschmann et al., (1992) J. Amer. Chem. Soc. 114: 92179218), analogous organic syntheses of small compound libraries (Chen et al. (1994) J. Amer. Chem. Soc. 116: 2661), oligocarbamates (Cho, et al., (1993) Science 261:1303), and/or peptidyl phospbonates (Campbell et al., (1994) J. Org. Chem. 59: 658). See, generally, Gordon et al., (1994) J. Med. Chem. 37:1385, nucleic acid libraries, peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like). In a particular embodiment of an assay of the invention, such a library comprises a large variety of analogs or derivatives of diamidines.

Generally, a plurality of assay mixtures are run in parallel with different agent concentrations to obtain a differential response to the various concentrations. Typically, one of these concentrations serves as a negative control, i.e., at zero concentration or below the level of detection.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St Louis, Mo., ChemStar, Ltd, Moscow, Ru, 3D Pharmaceuticals, Exton, Pa., Martek Biosciences, Columbia, Md., etc.).

Some assays for compounds capable of modulating Tdp1 activity are amenable to high throughput screening. High throughput screening systems are commercially available (see, e.g., Zymark Corp., Hopkinton, Mass.; Air Technical Industries, Mentor, Ohio; Beclaan Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high thruput and rapid start up as well as a high degree of flexibility and customization. The manufacturers of such systems provide detailed protocols the various high throughput.

### Treatment of Diseases

In one aspect, the invention provides a method of inhibiting Tdp1 activity in a subject. The method includes the step of administering to the subject a diamidine compound capable of modulating the activity of Tdp1.

In one preferred aspect, the administered diamidine compound comprises a furanyl moiety, such as a furanylene moiety.

In preferred embodiments, the diamidine compound is a compound of Formula I:

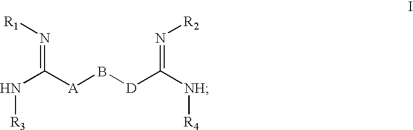

in which A, B and D are each independently C₁-C₆ alkylene, C₃-C₁₀ cycloalkylene, C₁-C₉ heterocycloalkylene, C₆-C₁₀arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In further preferred embodiments, A and D are each C₆-C₁₀ arylene and B is heteroarylene; more preferably, B is furanylene.

In one aspect, diamidine furan compounds of the following formula IA are provided:

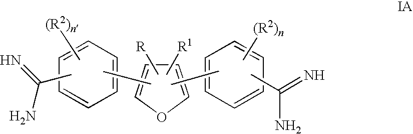

wherein R, R¹ and each R² are independently hydrogen or a non-hydrogen substituent such as halogen, hydroxyl, C₁₋₈alkylcarbonyloxy, C₅₋₁₅arylcarbonyloxy, C₁₋₈alkoxycarbonyloxy, C₅₋₁₅₈aryloxycarbonyloxy, C₁₋₈carboxylate, C₁₋₈alkylcarbonyl, C₁₋₈alkoxycarbonyl, C₁₋₈aminocarbonyl, C₁₋₈alkylthiocarbonyl, C₁₋₈alkoxyl, phosphate, phosphonato, phosphinato, cyano, amino (including C₁₋₈alkyl amino, C₁₋₈dialkylamino, C₅₋₁₅arylamino, C₅₋₁₅diarylamino, and C₅₋₁₅alkylarylamino), C₁₋₂₀acylamino (including C₁₋₈alkylcarbonylamino, C₅₋₁₅arylcarbonylamino, C₁₋₈carbamoyl and C₁₋₈ureido), amidino, imino, sulfhydryl, C₁₋₈alkylthio, C₅₋₁₅arylthio, C₁₋₈thiocarboxylate, sulfates, sulfonato, sulfamoyl, sulfonamido, nitro, trifluoromethyl, cyano, azido, C₁₋₁₂heterocyclyl, C₅₋₂₀alkylaryl, or an aromatic or heteroaromatic moiety;

n and n′ are each independently integers from 0 (where the phenyl ring does not have non-hydrogen R² substituents) to 4; and

pharmaceutically acceptable salts thereof.

In a preferred embodiment, the administered compound is one or both of the following or a pharmaceutically acceptable salt thereof:

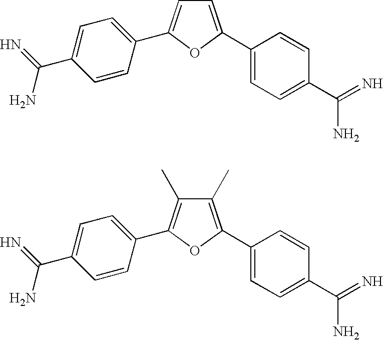

In another aspect, the invention provides a method of inhibiting Tdp1 activity in a subject identified as being in need of such treatment. The method includes the step of administering to the subject a diamidine compound, wherein the diamidine compound is capable of binding to Tdp1.

In another aspect, the invention provides a method treating a Tdp1-related disorder in a subject. The method includes the step of administering to the subject an effective amount of a diamidine compound, such that the subject is treated for the disorder, and the disorder is cancer, tumor, neoplasm, neovascularization, vascularization, cardiovascular disease, intravasation, extravasation, metastasis, arthritis, infection, Alzheimer's Disease, blood clot, atherosclerosis, melanoma, skin disorder, rheumatoid arthritis, diabetic retinopathy, macular edema, or macular degeneration, inflammatory and arthritic disease, or osteosarcoma.

In another aspect, the invention provides a method of treating cancer in a subject identified as in need of such treatment. The method includes the step of administering to the subject an effective amount of a compound of Formula I:

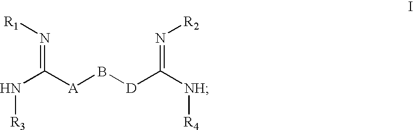

in which A, B and D are each independently C₁-C₆ alkylene, C₃-C₁₀ cycloalkylene, C₁-C₉ heterocycloalkylene, C₆-C₁₀ arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In further preferred embodiments, A and D are each C₆-C₁₀arylene and B is heteroarylene; more preferably, B is furanylene.

In one aspect, diamidine furan compounds of the following formula IA are provided:

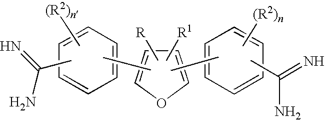

wherein R, R¹ and each R² are independently hydrogen or a non-hydrogen substituent such as halogen, hydroxyl, C₁₋₈ alkylcarbonyloxy, C₅₋₁₅arylcarbonyloxy, C₁₋₈alkoxycarbonyloxy, C₅₋₁₅₈aryloxycarbonyloxy, C₁₋₈carboxylate, C₁₋₈alkylcarbonyl, C₁₋₈alkoxycarbonyl, C₁₋₈aminocarbonyl, C₁₋₈alkylthiocarbonyl, C₁₋₈alkoxyl, phosphate, phosphonato, phosphinato, cyano, amino (including C₁₋₈alkyl amino, C₁₋₈ dialkylamino, C₅₋₁₅arylamino, C₅₋₁₅diarylamino, and C₅₋₁₅alkylarylamino), C₁₋₂₀acylamino (including C₁₋₈alkylcarbonylamino, C₅₋₁₅arylcarbonylamino, C₁₋₈carbamoyl and C₁₋₈ureido), amidino, imino, sulfhydryl, C₁₋₈alkylthio, C₅₋₁₅arylthio, C₁₋₈thiocarboxylate, sulfates, sulfonato, sulfamoyl, sulfonamido, nitro, trifluoromethyl, cyano, azido, C₁₋₁₂heterocyclyl, C₅₋₂₀alkylaryl, or an aryl (i.e. aromatic such as phenyl, etc.) or heteroaromatic moiety;

n and n′ are each independently integers from 0 (where the phenyl ring does not have non-hydrogen R² substituents) to 4; and

pharmaceutically acceptable salts thereof.

In a preferred embodiment, the administered compound selected from one or more of the following compounds or pharmaceutically acceptable salts thereof:

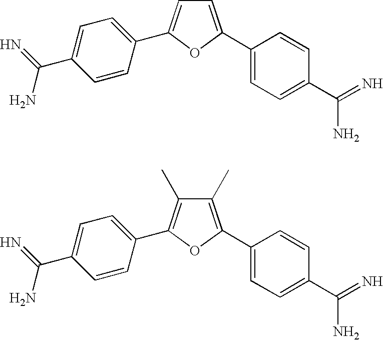

In further preferred embodiments, the compound is a Tdp1 inhibitor. In further preferred embodiments, the method further includes an additional therapeutic agent; preferably the additional therapeutic agent is an anticancer compound, more preferably a TopI inhibitor.

In further preferred embodiments, the step of administering the compound includes administering the compound orally, topically, parentally, intravenously or intramuscularly. In further preferred embodiments, the method includes the step of administering an effective amount of a composition including a diamidine compound and a pharmaceutically suitable excipient. In further preferred embodiments, the subject is a human.

In another aspect, the invention provides the use of a compound in the manufacture of a medicament for inhibiting or reducing cancer in a patient, the compound being of Formula I:

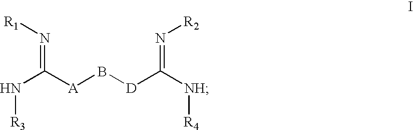

in which A, B and D are each independently C₁-C₆ alkylene, C₃-C₁₀ cycloalkylene, C₁-C₉ heterocycloalkylene, C₆-C₁₀ arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In another aspect, the invention provides a kit. The kit includes an effective amount of a diamidine compound according to the invention in unit dosage form, together with instructions for administering the compound to a subject suffering from cancer.

In still another aspect, the invention provides a method for identifying a compound that modulates the interaction of Tdp1 with a Tdp1 substrate. The method includes the steps of obtaining a crystal structure of Tdp1 or obtaining information relating to the crystal structure of Tdp1, in the presence and/or absence of a Tdp1 substrate, and modeling a test compound into or on the substrate binding site of the crystal structure to determine whether the compound modulates the interaction of Tdp1 with a substrate.

Tumors or neoplasms include new growths of tissue in which the multiplication of cells is uncontrolled and progressive. Some such growths are benign, but others are termed “malignant,” leading to death of the organism. Malignant neoplasms or “cancers” are distinguished from benign growths in that, in addition to exhibiting aggressive cellular proliferation, they invade surrounding tissues and metastasize. Moreover, malignant neoplasms are characterized in that they show a greater loss of differentiation (greater “dedifferentiation”), and of their organization relative to one another and their surrounding tissues. This property is also called “anaplasia.”

Neoplasms treatable by the present invention include all solid tumors, i.e., carcinomas and sarcomas, including Kaposi's sarcoma. Carcinomas include those malignant neoplasms derived from epithelial cells which tend to infiltrate (invade) the surrounding tissues and give rise to metastases. Adenocarcinomas are carcinomas derived from glandular tissue or in which the tumor cells form recognizable glandular structures. Sarcoma, including Kaposi's sarcoma broadly include tumors whose cells are embedded in a fibrillar or homogeneous substance like embryonic connective tissue.

The invention is particularly illustrated herein in reference to treatment of certain types of experimentally defined cancers. In these illustrative treatments, standard state-of-the-art in vitro and in vivo models have been used. These methods can be used to identify agents that can be expected to be efficacious in in vivo treatment regimens. However, it will be understood that the method of the invention is not limited to the treatment of these tumor types, but extends to any solid tumor derived from any organ system.

Thus, treatable cancers include, for example, colon cancer, bladder cancer, breast cancer, melanoma, ovarian carcinoma, prostatic carcinoma, or lung cancer, and a variety of other cancers as well. The invention is especially useful in the inhibition of cancer growth in adenocarcinomas, including, for example, those of the prostate, breast, kidney, ovary, testes, and colon. The invention is further useful against melanomas, which derive from the melanocytic system in the skin and other organs.

A solid tumor can be malignant, e.g. tending to metastasize and being life threatening, or benign. Examples of solid tumors that can be treated according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastorna, and retinoblastoma.

Moreover, tumors comprising dysproliferative changes (such as metaplasias and dysplasias) are treated or prevented in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

Other examples of tumors that are benign and can be treated with a method of the present invention include arteriovenous (AV) malformations, particularly in intracranial sites and myoleomas. A method of the present invention may also be used to treat psoriasis, a dermatologic condition that is characterized by inflammation and vascular proliferation; benign prostatic hypertrophy, a condition associated with inflammation and possibly vascular proliferation; and cutaneous fungal infections. Treatment of other hyperprobiferative disorders is also contemplated.

In certain embodiments, the present invention is directed to a method for inhibiting cancer growth, including processes of cellular proliferation, invasiveness, and metastasis in biological systems. The method includes the use of a compound of the invention (e.g., a diamidine compound) as an inhibitor of cancer growth. Preferably, the method is employed to inhibit or reduce cancer cell proliferation, invasiveness, metastasis, or tumor incidence in living animals, such as mammals.

The invention includes a method of inducing cytotoxicity (cell killing) in cancer cells or reducing the viability of cancer cells. For example, the invention can be used to induce cytotoxicity in cells of carcinomas of the prostate, breast, ovary, testis, lung, colon, or breast. The selective killing of the cancer cells can occur through apoptosis, necrosis, another mechanism, or a combination of mechanisms.

The killing of cancer cells can occur with less cytotoxicity to normal cells or tissues than is found with conventional cytotoxic therapeutics, preferably without substantial cytotoxicity to normal cells or tissues. For example, a compound of the invention can induce cytotoxicity in cancer cells while producing little or substantially no cytotoxicity in normal cells. Thus, unlike conventional cytotoxic anticancer therapeutics, which typically kill all growing cells, a compound of the invention can produce differential cytotoxicity: tumor cells may be selectively killed whereas normal cells may be spared. Thus, in another embodiment, the invention is a method for inducing differential cytotoxicity in cancer cells relative to normal cells or tissue. This differential in cytotoxicity associated with the compounds of the invention occurs as a result of apoptosis, necrosis, another mechanism, or a combination of such mechanisms.

In preferred embodiments, the compounds of the invention exhibit their cancer treatment properties at concentrations that lead to fewer side effects than those of known chemotherapeutic agents, and in highly preferred embodiments may be substantially free of side effects. The compounds of the invention are useful for extended treatment protocols, where other compounds would exhibit undesirable side-effects. In preferred embodiments, the properties of hydrophilicity and hydrophobicity are well balanced in these compounds, enhancing their utility both in vitro and especially in vivo, while other compounds lacking such balance are of substantially less utility. Thus, in preferred embodiments, the compounds will have an appropriate degree of solubility in aqueous media to permit absorption and bioavailability in the body, while also having a degree of solubility in lipids to permit traversal of the cell membrane to a putative site of action. The compounds are maximally effective if they can be delivered to the site of the tumor and are able to enter the tumor cells.

In the treatment of certain localized cancers, the degree of hydrophilicity of the compound can be of lesser importance. Compounds which have low solubility in aqueous systems, can be used in direct or topical treatment of skin cancers, e.g., melanoma or basal cell carcinoma, or by implantation into the brain to topically treat brain cancer.

In preferred embodiments, the compounds of the invention can inhibit the proliferation, invasiveness, or metastasis of cancer cells in vitro, as well as in vivo.

In certain preferred embodiments, the incidence or development of tumor foci can be inhibited or substantially prevented from occurring. Therefore, the methods of the invention can be used as a prophylactic treatment, e.g., by administering a compound to a mammal after detection of a gene product or metabolite associated with predisposition to a cancer but before any specific cancerous lesion is detected. Alternatively, the compounds are useful for preventing cancer recurrence, for example, to treat residual cancer following surgical resection or radiation therapy.

The amount of the compound used according to the invention is an amount that is effectively inhibitory of cancer growth. An amount of a compound is effectively inhibitory to cancer growth if it significantly reduces cellular proliferation or the potential of invasiveness or metastasis. Proliferation refers to the capacity of a tumor to increase its volume through cell division, typically measured as the “doubling rate.” The inhibition of cellular proliferation by the present method means that the rate of growth is decreased. In some cases the method can actually induce regression or diminution of tumor mass, if the rate of replenishment of the tumor cells through cell division is exceeded by the rate of cell death. Invasiveness refers to the potential of a tumor or tumor cells to invade other tissues, typically by breaking down the extracellular matrix of those tissues. Metastasis refers to the potential of a tumor or tumor cells to establish new tumor foci at sites distant from the primary site where the tumor began. Typically, metastasis proceeds by individual cells or groups of cells breaking off from the primary tumor and migrating, e.g., through the blood or lymph, to establish a new tumor focus in another tissue or organ. One locus common in tumor metastasis is in the lung, where the very fine vasculature of the lung tissue can often catch circulating tumor cells, permitting the establishment of a tumor focus therein. Some types of tumors metastasize to specific types of tissues.

The cancers treatable by means of the present invention occur in mammals. Mammals include, for example, humans, as well as pet animals such as dogs and cats, laboratory animals such as rats and mice, and farm animals such as horses and cows.

### Compounds Useful in the Methods of the Invention

Drug candidates encompass numerous chemical classes, though typically they are organic molecules, preferably small organic compounds having a molecular weight of more than 100 and less than about 2,500 daltons. Certain small molecules are less than 2000, or less than 1500 or less than 1000 or less than 500 D. Candidate agents typically include at least an amine, carbonyl, hydroxyl or carboxyl group, preferably at least two of the functional chemical groups. In certain preferred embodiments, the candidate agents are diamidines. In a preferred aspect, the candidate agents include diamidines that comprise a furanylene moiety.

In another aspect, the invention provides a compound of Formula I:

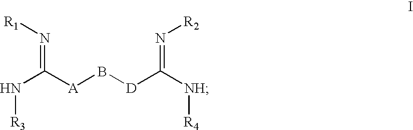

in which A, B and D are each independently C₁-C₆ alkylene, C₃-C₁₀ cycloalkylene, C₁-C₉ heterocycloalkylene, C₆-C₁₀ arylene, C₁-C₁₀ heteroarylene, or absent; R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted; R^{a} is independently for each occurrence H, alkyl, alkenyl, alkynyl, cycloalkyl, heterocycloalkyl, aralkyl, heteroaralkyl, aryl, heteroaryl, haloalkyl; —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted; or a pharmaceutically-acceptable salt thereof.

In certain preferred embodiments, the compound is identified by a method for identifying a compound which modulates the activity of a Tyrosyl-DNA phosphodiesterase (Tdp1).

In certain preferred embodiments of the compound, A and D are each C₆-C₁₀ arylene and B is heteroarylene; more preferably, B is furanylene.

In one aspect, diamidine furan compounds of the following formula IA are provided:

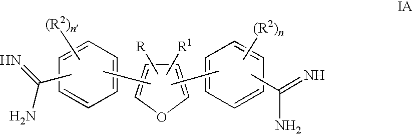

wherein R, R¹ and each R² are independently hydrogen or a non-hydrogen substituent such as halogen, hydroxyl, C₁₋₈alkylcarbonyloxy, C₅₋₁₅arylcarbonyloxy, C₁₋₈alkoxycarbonyloxy, C₅₋₁₅aryloxycarbonyloxy, C₁₋₈carboxylate, C₁₋₈alkylcarbonyl, C₁₋₈alkoxycarbonyl, C₁₋₈aminocarbonyl, C₁₋₈alkylthiocarbonyl, C₁₋₈alkoxyl, phosphate, phosphonato, phosphinato, cyano, amino (including C₁₋₈alkyl amino, C₁₋₈ dialkylamino, C₅₋₁₅arylamino, C₅₋₁₅diarylamino, and C₅₋₁₅alkylarylamino), C₁₋₂₀acylamino (including C₁₋₈alkylcarbonylamino, C₅₋₁₅arylcarbonylamino, C₁₋₈carbamoyl and C₁₋₈ureido), amidino, imino, sulfhydryl, C₁₋₈alkylthio, C₅₋₁₅arylthio, C₁₋₈thiocarboxylate, sulfates, sulfonato, sulfamoyl, sulfonamido, nitro, trifluoromethyl, cyano, azido, C₁₋₁₂heterocyclyl, C₅₋₂₀alkylaryl, or an aromatic or heteroaromatic moiety;

n and n′ are each independently integers from 0 (where the phenyl ring does not have non-hydrogen R² substituents) to 4; and

pharmaceutically acceptable salts thereof.

In a preferred embodiment, the compound is one or more of the following or a pharmaceutically acceptable salt thereof:

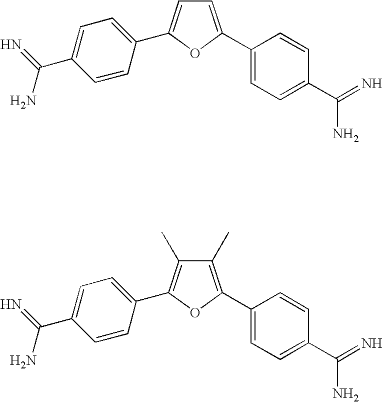

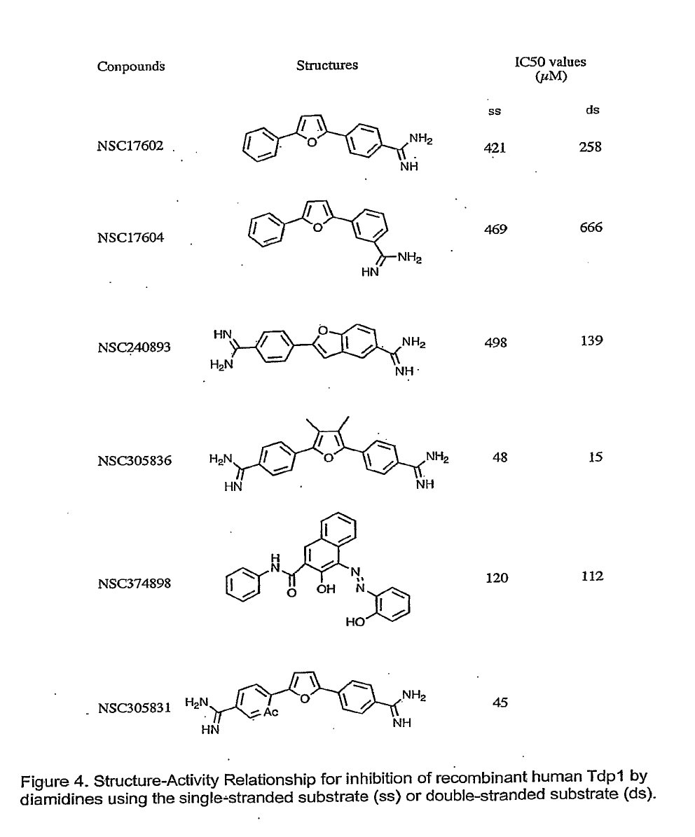

In preferred embodiments, the compound is a compound shown in FIG. 4. In certain preferred embodiments, of a compound of Formula I, each of R₁-R₄ is H.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Another aspect of the invention is a compound of any of the formulae herein for use in the treatment or prevention in a subject of a disease, disorder or symptom thereof delineated herein. Another aspect of the invention is use of a compound of any of the formulae herein in the manufacture of a medicament for treatment or prevention in a subject of a disease, disorder or symptom thereof delineated herein.

Compounds as disclosed herein can be readily prepared by known synthetic procedures. For instance, a halogenated furan can be reacted with an appropriately substituted aryl compound such as a substituted phenyl compound to couple the reagents. For instance, a halogenated group (such as a furan with one or more bromo ring substituents) can be coupled with an aryl group (such as a phenyl group) via a Stile Coupling or Mitsunobu Coupling (e.g. where a tin-aryl reagent is reacted with the halo-reagent). Suitable coupled groups (such as a furan coupled to one or more aryl including phenyl groups) also are commercially available. A nitrogen-containing ring substituent of the aryl moiety (e.g. phenyl) such as cyano, alkyl amine or the like can be functionalized to provide an amidine moiety.

### Formulation, Administration, and Pharmaceutical Compositions

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

The phrase “pharmaceutically acceptable” is refers to those compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, involved in carrying or transporting the subject chemical from one organ, or portion of the body, to another organ, or portion of the body. Each carrier must be “acceptable” in the sense of being compatible with the other ingredients of the formulation and not injurious to the patient.

A therapeutically effective amount can be administered in one or more doses. The term “administration” or “administering” includes routes of introducing the compound(s) to a subject to perform their intended function. Examples of routes of administration which can be used include injection (subcutaneous, intravenous, parenterally, intraperitoneally, intrathecal), oral, inhalation, rectal and transdermal.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

Methods of preparing these compositions include the step of bringing into association a compound(s) with the carrier and, optionally, one or more accessory ingredients. These compositions may also contain adjuvants such as preservatives, wetting agents, emulsifying agents and dispersing agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. Alternatively, delayed absorption of a parenterally-administered drug form is accomplished by dissolving or suspending the drug in an oil vehicle.

Regardless of the route of administration selected, the compound(s), which may be used in a suitable hydrated form, and/or the pharmaceutical compositions of the present invention, are formulated into pharmaceutically-acceptable dosage forms by conventional methods known to those of skill in the art.

In certain embodiments, the pharmaceutical compositions are suitable for topical, intravenous, intratumoral, parental, or oral administration. The methods of the invention further include administering to a subject a therapeutically effective amount of a conjugate in combination with another pharmaceutically active compound. Pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference.

Formulations are provided to a subject in an effective amount. The term “effective amount” includes an amount effective, at dosages and for periods of time necessary, to achieve the desired result. An effective amount of conjugate may vary according to factors such as the disease state, age, and weight of the subject, and the ability of the compound to elicit a desired response in the subject. Dosage regimens may be adjusted to provide the optimum therapeutic response.

The effective amount is generally determined by the physician on a case-by-case basis and is within the skill of one in the art. As a rule, the dosage for in vivo therapeutics or diagnostics will vary. Several factors are typically taken into account when determining an appropriate dosage. These factors include age, sex and weight of the patient, the condition being treated, and the severity of the condition.

Suitable dosages and formulations of immune modulators can be empirically determined by the administering physician. Standard texts, such as Remington: The Science and Practice of Pharmacy, 17th edition, Mack Publishing Company, and the Physician's Desk Reference, each of which are incorporated herein by reference, can be consulted to prepare suitable compositions and doses for administration. A determination of the appropriate dosage is within the skill of one in the art given the parameters for use described herein. Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

In terms of treatment, an effective amount is an amount that is sufficient to palliate, ameliorate, stabilize, reverse or slow the progression of a cancerous disease or otherwise reduce the pathological consequences of the cancer. A therapeutically effective amount can be provided in one or a series of administrations. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the compound being administered.

Ascertaining dosage ranges is well within the skill of one in the art. The dosage of compounds of the invention can range from, e.g. about 0.001 μg/kg body weight/day to 500 mg/kg body weight/day, preferably about 1 μg/kg/day to 100 mg/kg/day, still more preferably about 110 μg/kg/day to 50 mg/kg/day. Methods for administering compositions are known in the art. Such dosages may vary, for example, depending on whether multiple administrations are given, tissue type and route of administration, the condition of the individual, the desired objective and other factors known to those of skill in the art. Administrations can be conducted infrequently, or on a regular weekly basis until a desired, measurable parameter is detected, such as diminution of disease symptoms. Administration can then be diminished, such as to a biweekly or monthly basis, as appropriate.

Such dosages may vary, for example, depending on whether multiple administrations are given, tissue type and route of administration, the condition of the individual, the desired objective and other factors known to those of skill in the art

Following administration of the composition, it can be necessary to wait for the composition to reach an effective tissue concentration at the site of the disorder before detection. Duration of the waiting step varies, depending on factors such as route of administration, location, and speed of movement in the body. In addition, where the compositions are coupled to molecular carriers, the rate of uptake can vary, depending on the level of receptor expression on the surface of the cells. For example, where there is a high level of receptor expression, the rate of binding and uptake is increased. Determining a useful range of waiting step duration is within the level of ordinary skill in the art and may be optimized.

Within broad limits, the compounds of the invention are expected to exhibit dose-dependent effects; therefore, administration of larger quantities of a compound is expected to inhibit cancer cell growth or invasiveness to a greater degree than does administration of a smaller amount. In preferred embodiments, debilitating side effects usually attendant upon conventional cytotoxic cancer treatments are reduced, and preferably avoided.

Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with other pharmaceutical agents.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow-release or sustained release forms, whereby a relatively consistent level of the active compound are provided over an extended period.

Another method of administration is intravascular, for instance by direct injection into the blood vessel, or surrounding area. Further, it may be desirable to administer the compositions locally to the area in need of treatment; this can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel® provided by Guilford Pharmaceuticals Inc.

Although methods and materials similar or equivalent to those described herein can be used in the practice of the present invention, preferred methods and materials are described above. The materials, methods, and examples are illustrative only and not intended to be limiting. Other features and advantages of the invention will be apparent from the detailed description and from the claims.

Enteral administration is a preferred route of delivery of the compound of the invention, and compositions including the compound with appropriate diluents, carriers, and the like are readily formulated. Liquid or solid (e.g., tablets, gelatin capsules) formulations can be employed. It is among the advantages of the invention that, in many situations, the compound can be delivered orally, as opposed to parenteral delivery (e.g., injection, infusion) which is typically required with conventional chemotherapeutic agents.

Parenteral use (e.g., intravenous, intramuscular, subcutaneous injection) is also contemplated, and formulations using conventional diluents, carriers, etc., such as are known in the art can be employed to deliver the compound.

Alternatively, delivery of the compound can include topical application. Compositions deemed to be suited for such topical use include as gels, salves, lotions, ointments and the like. In the case of tumors having foci inside the body, e.g., brain tumors, the compound of the invention can be delivered via a slow-release delivery vehicle, e.g., a polymeric material, surgically implanted at or near the lesion situs.

The maximal dosage for a subject is the highest dosage that does not cause undesirable or intolerable side effects. In any event, the practitioner is guided by skill and knowledge in the field, and the present invention includes, without limitation, dosages that are effective to achieve the described phenomena.

The invention can also be practiced by including with the compound one or more other anti-cancer chemotherapeutic agents, such as any conventional chemotherapeutic agent. The combination of the compound with such other agents can potentiate the chemotherapeutic protocol. Numerous chemotherapeutic protocols will present themselves in the mind of the skilled practitioner as being capable of incorporation into the method of the invention. Any chemotherapeutic agent can be used, including alkylating agents, antimetabolites, hormones and antagonists, radioisotopes, as well as natural products. For example, the non-anti-microbial compound of the invention can be administered with antibiotics such as doxorubicin and other anthracycline analogs, nitrogen mustards such as cyclophosphamide, pyrimidine analogs such as 5-fluorouracil, cisplatin, hydroxyurea, taxol and its natural and synthetic derivatives, and the like. As another example, in the case of mixed tumors, such as adenocarcinomas of the breast and prostate, in which the tumors can include gonadotropin-dependent and gonadotropin-independent cells, the compound of the invention can be administered in conjunction with leuprolide or goserelin (synthetic peptide analogs of LH-RH). Other antineoplastic protocols include the use of a compound of the invention with another treatment modality, e.g., surgery, radiation, other chemotherapeutic agent, etc., referred to herein as “adjunct antineoplastic modalities.” Thus, the method of the invention can be employed with such conventional regimens with the benefit of reducing side effects and enhancing efficacy.

Human cancers are characterized by genomic instability, which leads to the accumulation of DNA lesions. Hence, tumor cells are highly dependent on normal repair for survival.

DNA topoisomerase I (Top1) is ubiquitous and essential in higher eukaryotes. It relieves DNA torsional stress and relaxes DNA supercoiling by introducing DNA single-strand breaks. Top1 can be trapped by DNA lesions that accumulate in cancer cells. Top1 is also the target of the anticancer agent camptothecin and non-camptothecin inhibitors. Top1 inhibitors damage DNA by trapping covalent complexes between the Top1 catalytic tyrosine and the 3′-end of the broken DNA. Tyrosyl-DNA phosphodiesterase (Tdp1) repairs Top1-DNA covalent complexes by hydrolyzing the tyrosyl-DNA bond.

Tdp1 inhibitors are therefore useful as anticancer agents both in monotherapy and in combination with other anticancer compounds (particularly DNA-targeted anticancer compounds) such as Top1 inhibitors. Tumor cells, whose repair pathways are commonly deficient, might be selectively sensitized to Top1 inhibitors compared to normal cells that contain redundant repair pathways. Moreover, Tdp1 inhibitors might also be effective by themselves as anticancer agents as oncogenic activation tends to increase free radical production and genomic instability (Cerutti P A (1985) Science 227 (4685):375-381; Kc S et al. Mutat Res. (2006) 29 593(1-2):64-79.; Vafa et al., Mol Cell 9(5):1031-1044 (2002)).

Thus, in certain embodiments, the invention provides methods for treating cancer and other cell proliferative disorders by administering to a subject in need thereof an effective amount of a combination of a Tdp1 inhibitor of this invention together with a TopI inhibitor. A variety of TopI inhibitors have been reported, including camptothecin, irinotecan, topotecan, saintopin, and derivatives and analogs thereof. In another aspect, the invention provides pharmaceutical compositions including a Tdp1 inhibitor of this invention together with a TopI inhibitor, optionally including a pharmaceutically-acceptable carrier or excipient.

In another aspect, the invention provides methods and compositions for the treatment or prevention of parasitic disease. Tdp1 inhibitors may be valuable as anti-infectious agents since the gene is present in parasites, including Trypanosoma brucei rhodesiense, Trypanosoma brucei gambiense, and Plasmodium spp. including P. vivax, P. falciparum, P. ovale, and P. malaria. Thus, in one aspect, the invention provides methods for treating or preventing a parasitic infection caused by a parasite expressing Tbp1, the method including the step of administering to a subject in need thereof an effective amount of a Tdp1 inhibitor according to this invention. In another aspect, the invention provides pharmaceutical compositions for treatment or preention of parasitic disease, including a Tdp1 inhibitor of this invention together pharmaceutically-acceptable carrier or excipient.

### EXAMPLES

Tdp1 inhibitors have become a major area of drug research and structure-based design, with Tdp1, works synergistically and selectively in the cancer cells. Tdp1 can repair DNA topoisomerase I (Top1) covalent complexes by hydrolyzing the tyrosyl-DNA phosphodiester bond. The natural substrate of Tdp1 is large and complex, consisting of tyrosine or possibly a tyrosine-containing peptide moiety linked to a single strand of DNA via a 3′ phosphodiester bond (Interthal, H.; Pouliot, J. J. PNAS, 98, 21 (2001)). In the present study, in order to determine how the inhibitors may be binding with the active site of Tdp1 N domain, we report docking the inhibitors into a structural model of Tdp1 enzyme, based on a multiple crystal structures of Tdp1 substrate complex with resolution 2.0 or better inhibitors to obtain information about their preferred conformations and their potential binding interactions with the Tdp1 and Top1 N-terminal domain.

### Materials and Methods

Computational modeling was performed using Glide software (Schrodinger Inc.) on a Silicon Graphics workstation. All minimizations and docking were performed with the OPLS2003 force field. The dimmer complex with peptide [1NOP (Davies D. R., Champoux J. J., J. Med. Chem., 324,917-932, 2002), 1RFF (Davies, D. R. et al., Chem. & Biol. 10, 139 (2003))] and with octopamine (1RHO) (Davies, D. R. et al., J. Med. Chem. 47, 829 (2004)) were used. Chain A from the crystal structure of Top1 and Tdp1 bound to the NT domain was used as the starting geometry for the modeling study. The model was built from an x-ray crystal structure of the complexes: 1NOP, 1RFF, 1RHO using the Maestro 7.5.

### Example 1

Eight crystal structures (shown in Table 1, Davies, D. R. et al., J. Med. Chem. 47, 829 (2004)) of Tdp1 with vanadate, oligonucleotides and peptides or peptide analogues were determined. Those eight complex include peptides of varying length and sequence, non-peptide analogues of tyrosine and oligonucleotides of varying length of sequence. The conformations of the 8WT (Top1-peptide) and eight other crystallographic peptides in vanadate complex with Tdp1 are significantly different from the conformation of the corresponding residues in the crystal structures of Top1 bound to DNA (1NOP) (Davies, D. R., et al., Structure 10 237 (2002))

### Example 2

Two compounds were used, as shown below by structures (a) and (b):

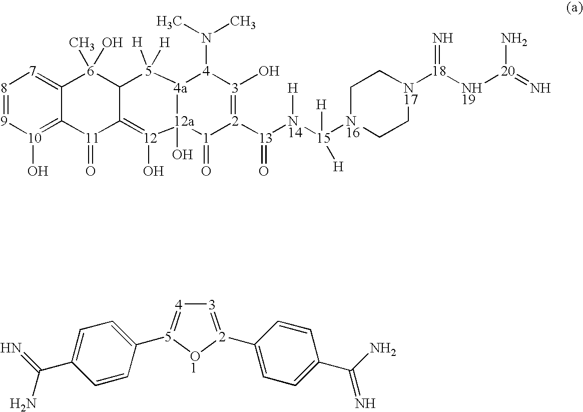

(b) (2,5-di-(4-phenylamidine)furan)

The data for the two compounds is as follows:

(a) IC₅₀ ss14Y=12 μM; IC₅₀ ds14Y=19 μM; MW=626.66
(b) IC₅₀ ss14Y=45 μM; IC₅₀ ds14Y=13.2 μM; MW=304.35

The two dimensional structures of Compounds (a) and (b) were minimized before analyzing the interactions between the ligand and the receptor. The compounds were optimized using the OPLS2003 force field, using a PRCG to convergence and a distance dependent dielectric constant of 1 for the electrostatic treatment. Minimization was done using conjugate gradient minimization. Maximum number of cycles was set to 1000, gradient criteria: 0.001. The complex was modeled in the N-terminal domain. The ligand compounds were docked by standard precision (SP) and with option: dock flexibly, which allow flips of 5 and 6 member rings. The best poses of compounds were finally selected based on the docking score, Emodel and the interactions made by the compounds with the active site of Tdp1.

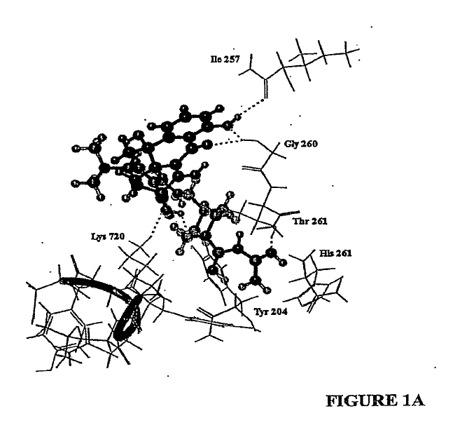

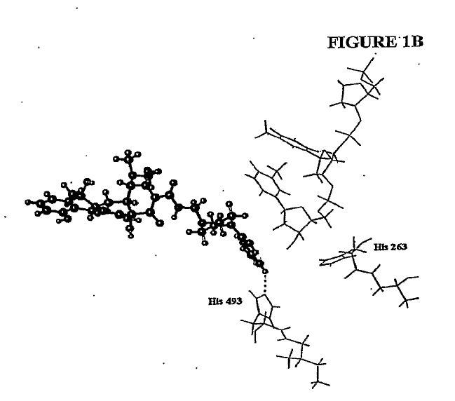

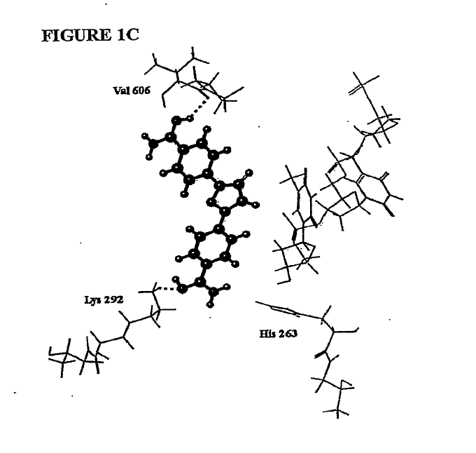

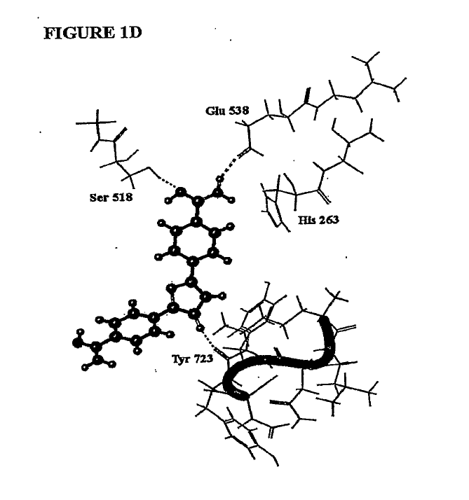

The results are shown in FIGS. 1-3. The docking analysis indicate that in the best poses of Compound (a) (without substrate): the amine group Hydrogen bonds to residue SER 608 (1NOP); hydrogen atom of imine group to His 493 (1RFF); nitrogen atom by C20 contacts with polar hydrogen atom of T 806 of oligonucleotide (1RHO) and in the best poses of Compound (a) (with substrate): oxygen atom of hydroxyl group by C10 and carbonyl group by C11 HBonds to GLY 260, oxygen atom of hydroxyl group by C12 to LYS 720 (1RFF); and the hydrogen atom of hydroxyl group by C12a of NSC 118695 HBonds to PRO461 (1RHO). In case of Compound (b) (without substrate) hydrogen atom of amine group HBonds to N3 of His 493 (1RHO); nitrogen atom of imine group contacts with T 806 (1RHO), while in crystal structure oxygen atom of VO4 bonds to T 806 of oligonucleotide. When the substrate is present in the active site the amine group contacts with N3 and C4 of His 263 (1NOP); substituent in position 3 contacts with TYR 723 (1RFF); hydrogen atom of amine group contacts to N3 of His 263 (1RHO). The binding model obtained for Compound (a) did not show some critical interactions with active sites of Tdp1 N domain. The docking scores correlated poorly with enzyme inhibitory activity, so other approaches were tried to quantify the docked poses. One main reason for the failure to get a good docking mode could be attributed to the size of the molecules and the number of rotatable bonds (13 for Compound (a) and only 4 for Compound (b)).

### Example 3

The results obtained in Examples 1 and 2 are used to refine a model for prediction of binding affinity of compounds against Tdp1 as follows.

Virtual screening method of compounds obtained from, e.g., the NCI databases such as ChemNavigator based on the biological activity data of confirmed 34 compounds active in the low micromolar range. The obtained compounds will then be subjected to flexible docking as described above, and compounds are selected based on the docking score for the Energy of the Model. The results are compared with a training set of compounds found to bind to the active site of Tdp1.

### Example 4

Preparation of Tdp1 substrates: HPLC purified oligonucleotides N14Y (Plo et al., (2003) DNA Repair (Amst) 2(10):1087-1100) were labeled at their 5′-end with [γ-³²P]-ATP (Perkin-Elmer Life Science Co., Boston, Mass.) by incubation with 3′-phosphatase free T4 polynucleotide kinase (Roche applied Science, Indianapolis, Ind.) according to the manufacturer's protocols. Unincorporated nucleotides were removed by Sephadex G-25 spin-column chromatography (Mini Quick Spin Oligo Columns, Roche, Indianapolis, Ind.). For the production of the oligonucleotide duplexes D14Y, N14Y was mixed with the complementary oligonucleotide in equal molar ratios in annealing buffer (10 mM Tris-HCl pH 7.5, 100 mM NaCl, 10 mM MgCl₂), heated to 96° C., and allowed to cool down slowly (over 2 h) to room temperature.
Tdp1 assays: Unless indicated otherwise, Tdp1 assays are performed in 20 μl mixtures containing 50 mM Tris-HCl, pH 8.0, 80 mM KCl, 2 mM EDTA, 1 mM dithiothreitol (D)T, and 40 μg/ml bovine serum albumin (BSA). For initial screening of Tdp1 inhibitors, 25 nM of 5′-³²P-labeled substrate (D14Y) is reacted with 1 ng Tdp1 (≈0.7 nM) in the absence or presence of inhibitor for 20 min at 25° C. Reactions are stopped by addition of 60 μl of gel loading buffer (98% (v/v) formamide, 1% (w/v) xylene cyanol, 1% (w/v) bromophenol blue). Twelve μl of aliquots are resolved in 20% denaturing polyacrylamide (AccuGel, National Diagnostics, Atlanta, Ga.) (19:1) gel containing 7 M urea. After drying, gels are exposed overnight to PhosphorImager screens (Molecular Dynamics, Sunnyvale, Calif.). Screens are scanned, and images are obtained with the Molecular Dynamics software (Sunnyvale, Calif.). Densitometry analyses are performed using ImageQuant 5.2 software package (Amersham Biosciences, Piscataway, N.J.). Tdp1 activity is determined by measuring the fraction of substrate converted into 3′-phosphate DNA product by densitometry analysis of the gel image (Debethune L, Kohlhagen G, Grandas A and Pommier Y (2002) Nucleic Acids Res 30(5):1198-1204).
Results: FIG. 4 shows the structures of certain compounds, including amidines and diamidines, that were screened for activity against Tdp1. It can be seen that diamidines were the most potent inhibitory compounds in this group.

### Example 5

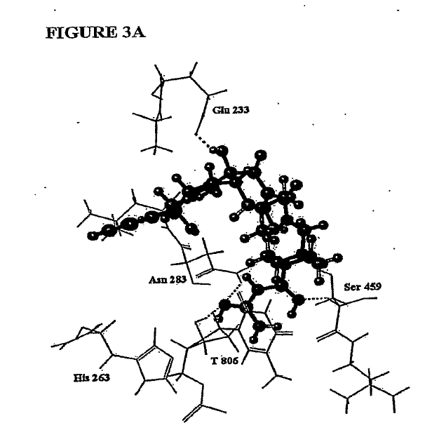

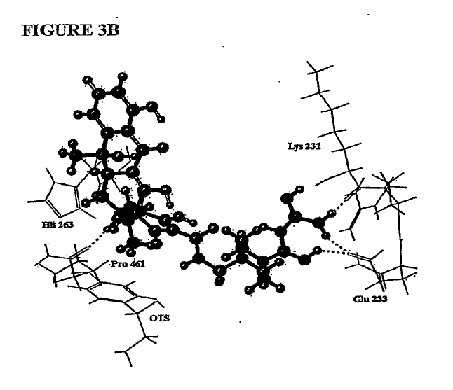

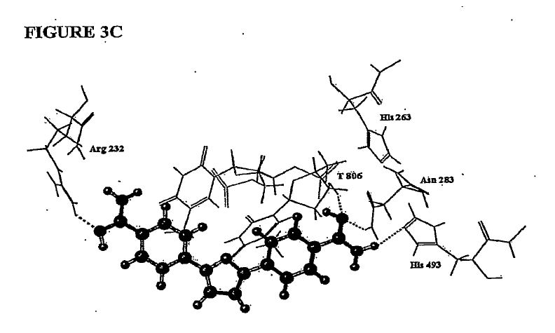

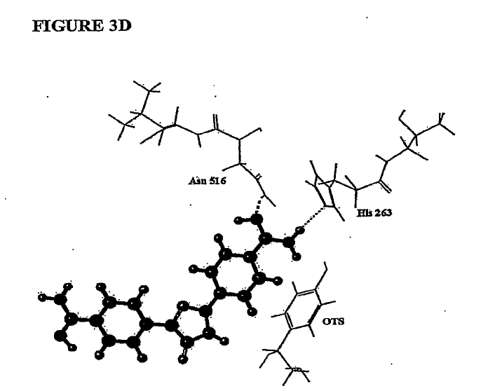

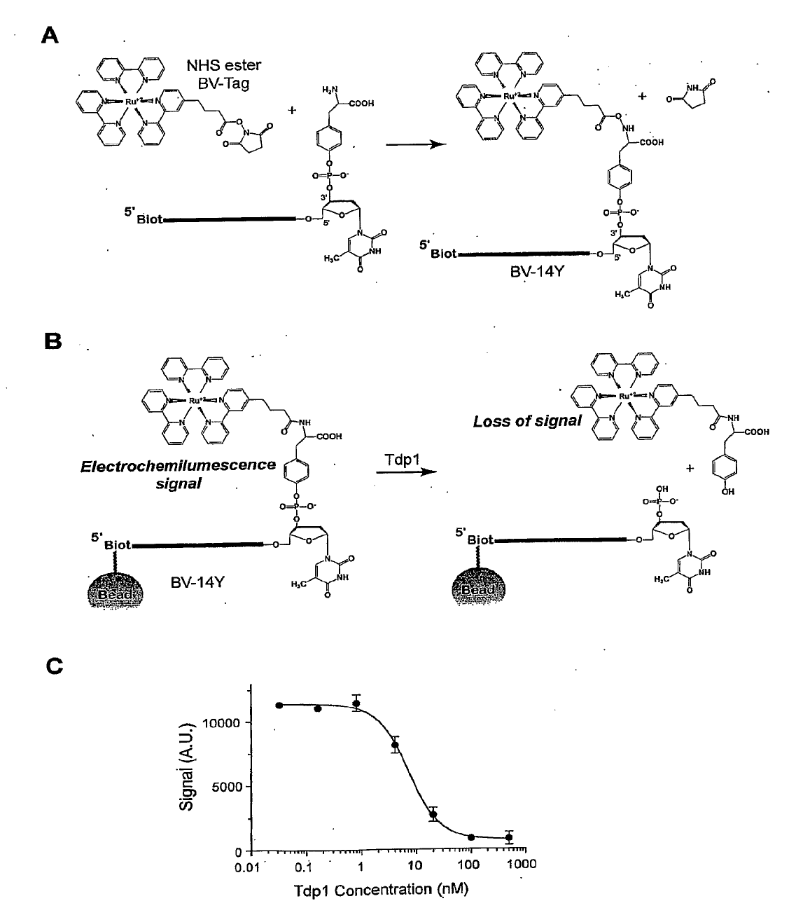

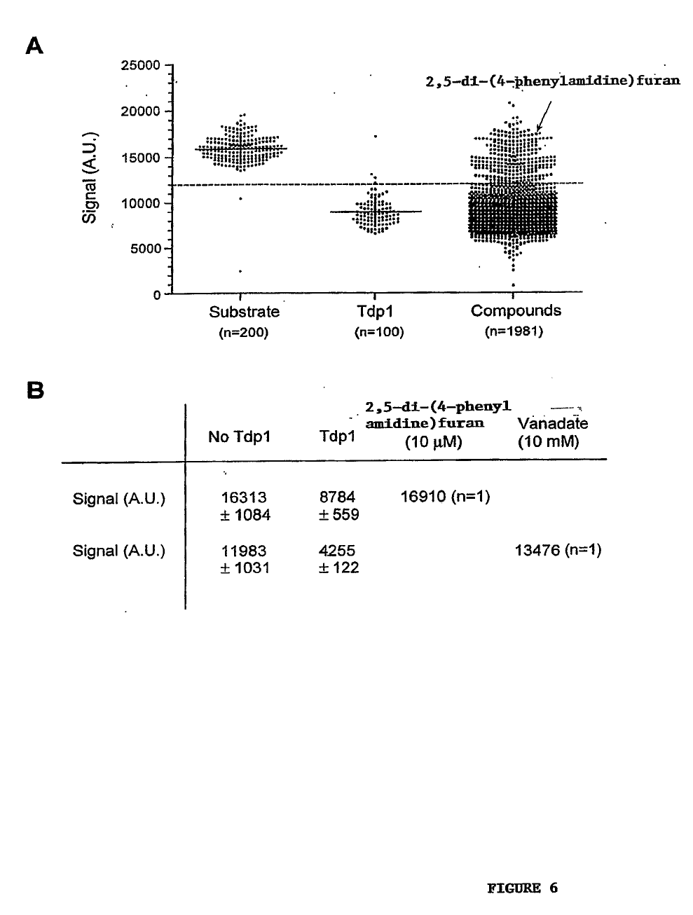

Drugs and Reagents: 2,5-di-(4-phenylamidine)furan and the 1980 compounds of the diversity set were from drug therapeutics development (DTP), NCI, NIH. Berenil and Pentamidine were from Sigma-Aldrich (St. Louis, Mo.). High-performance liquid chromatography-purified oligonucleotides were purchased from the Midland Certified Reagent Co. (Midland, Tex.).
Preparation of Human Tdp1: Human Tdp1 expressing plasmid pHN1910 (a gift from Dr. Howard Nash, Laboratory of Molecular Biology, National Institute of Mental Health, National Institutes of Health) was constructed using vector pET 15b (Novagen, Madison, Wis.) with full-length human Tdp1 and an additional Histag sequence of MGSSHHEHHSSGLVPRGSHMLEDP in its N terminus. The His-tagged human Tdp1 was purified from Novagen BL21 cells using chelating Sepharose™ fast flow column (Amersham Biosciences, Sweden) according to the company's protocol. Samples were assayed immediately. Tdp1 fractions were pooled and dialyzed with dialysis buffer (20% glycerol, 50 mM Tris-HCI, pH 8.0, 100 mM NaCl, 10 mM (β-mercaptoethanol, and 2 mM EDTA). Dialyzed samples were aliquoted and stored at −80° C. Tdp1 concentration was determined using Bradford protein assay (Bio-Rad Laboratories, Hercules, Calif.), and its purity was analyzed by SDS-polyacrylamide gel electrophoresis (SDS-PAGE).
High-throughput electrochemiluminescent assay: The electrochemiluminescent (ECL) assay utilized was based on the BioVeris (3V) ECL technology developed by BioVeris, Inc. (Gaithersburg, Md.). The ECL is based on the use of ruthenium labels (BV-TAG™′″), designed to emit light when stimulated. These labels, together with a specific instrumentation (M-series Analyzer), provide a novel platform for biological measurements.
Preparation of the ECL substrate: The 5′-biotinylated 14Y DNA substrate (sequence shown in FIG. 3A) was obtained from Midland Certified Reagent and coupled to an NHS ester BV-Tag (BioVeris Inc.) to generate the ECL substrate BV-14Y. Coupling was achieved by incubating 175 μl of 5′-biotinylated 14Y DNA at 200 μM in phosphate buffered saline (PBS), pH 7.4 with 25 μl of NHS-ester BV-Tag (BioVeris Inc.) at 3 μg/μl in 100% DMSO. After 30 min at room temperature under agitation the coupling reaction was loaded onto an Oligo spin column (Roche Diagnostics, Indianapolis, Ind.) pre-equilibrated with 3 volumes of PBS, pH 7.4 containing 0.075% (w/v) sodium azide (Sigma-Aldrich, St. Louis, Mo.). The recovered fraction was aliquoted and stored at −20° C. at 10 μM in PBS. ECL assay: Linking of the ECL BV-14Y substrate to streptavidin magnetic beads (Dynabeads M-280, BioVeris Inc.) was performed prior to the assay reaction following the manufacturer's instructions. The ECL BV-14Y substrate bound to the magnetic beads at a concentration of 0.8 nM was incubated with 1 nM Tdp1 in the absence or presence of 10 μM drug to be tested at a final volume of 100 μl/well in a 96-well plate format. The catalytic reaction was carried out in a buffer containing 50 mM Tris-HCI pH 8.0, 80 mM KCI, 2 mM EDTA and 1 mM DTT at room temperature for 60 min. Reactions were stopped by adding 1 volume of stop buffer (25 mM MES pH 6.0, 0.5% SDS). Plates were read on a M-Series M8 analyzer (BioVeris Inc.) and the ECL arbitrary units were plotted using the Prism software (Graphpad).
Preparation of Tdp1 Substrates for gel assays. As described in Example 4 above, high-performance liquid chromatography-purified oligonucleotides 14Y (see FIG. 3A) (Plo et al., 2003) and 14Y-CC (see FIG. 5A) were labeled at their 5′-end with [γ-³²P]ATP (PerkinElmer Life and Analytical Sciences, Boston, Mass.) by incubation with 3′-phosphatase-free T4 polynucleotide kinase (Roche Diagnostics, Indianapolis, Ind.) according to the manufacturer's protocols. Unincorporated nucleotides were removed by Sephadex G-25 spin-column chromatography (Mini Quick Spin Oligo columns; Roche Diagnostics). For the production of the oligonucleotide duplexes D14Y, radiolabeled 14Y was mixed with the complementary oligonucleotide (see FIG. 3A) in equal molar ratios in annealing buffer (10 mM Tris-HCl, pH 7.5, 100 mM NaCl, and 10 mM MgCl2), heated to 96° C., and allowed to cool down slowly (over 2 h) to room temperature.
Tdp1 gel assays: As described in Example 4 above, unless indicated otherwise, Tdp1 assays were performed in 20 μl mixtures containing 50 mM Tris-HCI, pH 8.0, 80 mM KCI, 2 mM EDTA, 1 mM dithiothreitol, and 40 μg/ml bovine serum albumin. For the assay, 25 nM 5′ ³²P-labeled substrate (14Y or 14Y-CC or D14Y) was reacted with 1 ng of Tdp1 (0.7 nM) in the absence or presence of inhibitor for 20 min at 25° C. Reactions were stopped by the addition of 60 μl of gel loading buffer [96% (v/v) formamide, 10 mM EDTA, 1% (w/v) xylene cyanol, and 1% (w/v) bromphenol blue]. Twelve microliter aliquots were resolved in 20% denaturing polyacrylamide (AccuGel; National Diagnostics, Atlanta, Ga.) (19:1) gel containing 7 M urea. After drying, gels were exposed overnight to Phosphorimager screens (GE Healthcare). Screens were scanned, and images were obtained with the Molecular Dynamics software. Densitometry analyses were performed using ImageQuant 5.2 software package (GE Healthcare). Tdp1 activity was determined by measuring the fraction of substrate converted into 3′-phosphate DNA product by densitometry analysis of the gel image (Debethune et al., 2002). Figures show representative results that were consistently reproduced at least three times.
Surface plasmon resonance analysis: Binding experiments were performed on a Biacore 2000 instrument (Biacore Inc., Piscatawy N.J.). 5′ biotinylated stem-loop (biotin-GATCTAAAAGACTTTCTCAAGTCTTTTAGATC) and single-stranded oligonucleotides (biotin-GATCTAAAAGACTT) were synthesized by IDT (Coralville, Iowa). Stem-loop oligonucleotides were annealed by heating to 90° C. for 5 min followed by snap cooling on ice for 15 min. Biotinylated oligonucleotides were immobilized to neutravidin-coated sensor chips as described previously (Fisher et al., 2006). Approximately 5000 RU's of neutravidin was attached to all flow cells on the sensor chips. Oligonucleotides were reconstituted in buffer consisting of 10 mM Tris (pH 7.5), 300 mM NaCl and 1 mM EDTA. Singlestranded and stem-loop oligonucleotides were injected over flow cell 2 and 4 respectively until approximately 500 RU's of were captured on the chip surface. 2,5-di-(4-phenylamidine)furan was diluted into running buffer (10 mM MES, 100 mM NaCl, 1 mM EDTA, 5% (v/v) pH 6.25) and injected over all flow cells at 20 ml/min at 25° C. Following compound injections, the surface was regenerated with a 10 second 1 M NaCl injection followed by a 10 second running buffer injection. A DMSO calibration curve was included to correct for refractive index mismatches between the running buffer and compound dilution series. Data was analyzed using the Scrubber software version 2 and the equilibrium binding of 2,5-di-(4-phenylamidine)furan was fit to either a single-site or two-site steady state binding model.
Results: Novel high-throughput electrochemiluminescent (ECL) assay to screen for Tdp1 inhibitors. To discover inhibitors of Tdp1, we developed a novel ECL high-throughput assay. See FIG. 5. An ECL substrate for Tdp1 was generated after coupling with a ruthenium containing tag (BV tag) as shown in FIG. 5A. In the presence of recombinant Tdp1 enzyme, the ECL substrate (BV-14Y DNA) is processed leading to the removal of the tyrosyl-BV-Tag group and therefore to a loss of signal (FIGS. 5B and 5C). A potential Tdp1 inhibitor would prevent this loss of signal. The level of signal retention would be reflective of the potency of the putative Tdp1 inhibitor. In our high-throughput screening system, any compound that did not restore the signal lost in the presence of Tdp1 to greater than 50% was considered inactive. Of the 1981 compounds screened at a single concentration of 10 mM, most of them were inactive in inhibiting Tdp1 activity (represented by the dots below the horizontal line in FIG. 6A). Of the remaining compounds, 169 were active at inhibiting Tdp1 activity by 70% or more (signal value <14,054). Subsequent analysis of the purity of the compounds by HPLC reduced the number of potential inhibitors of Tdp1 from 169 to 69. Counter screening in our gel based assay confirmed 49 compounds to inhibit Tdp1 activity to varying degrees (data not shown). The dication 2,5-di-(4-phenylamidine)furan is a potent inhibitor of Tdp1, that restores the signal lost in the presence of Tdp1 (for values, see table in FIG. 6B). For comparison, activity of the previously described inhibitor of Tdp1 (Davies et al., 2002), vanadate at 10 mM is shown. Thus, the ECL high-throughput assay is a sensitive and reliable technique for the screening of novel Tdp1 inhibitors.

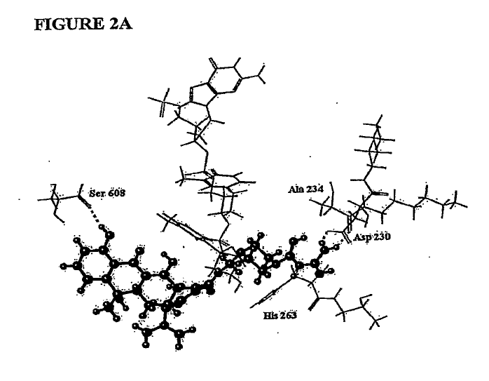

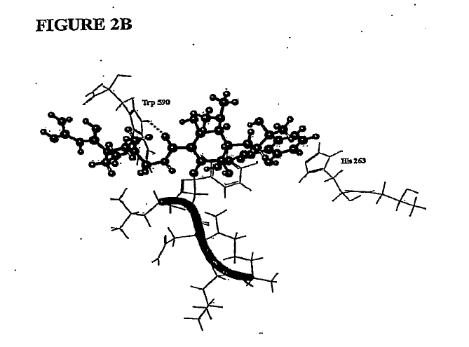

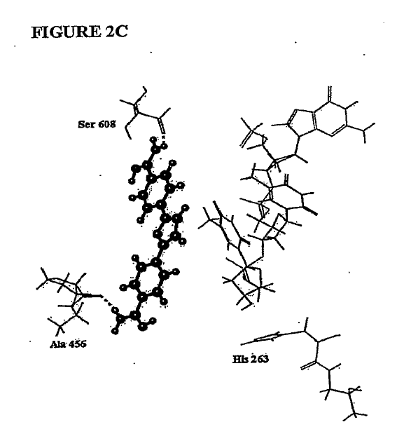

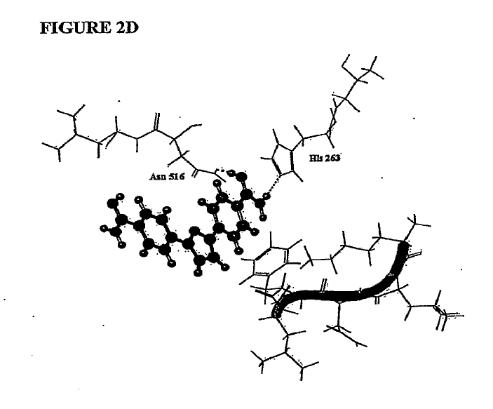

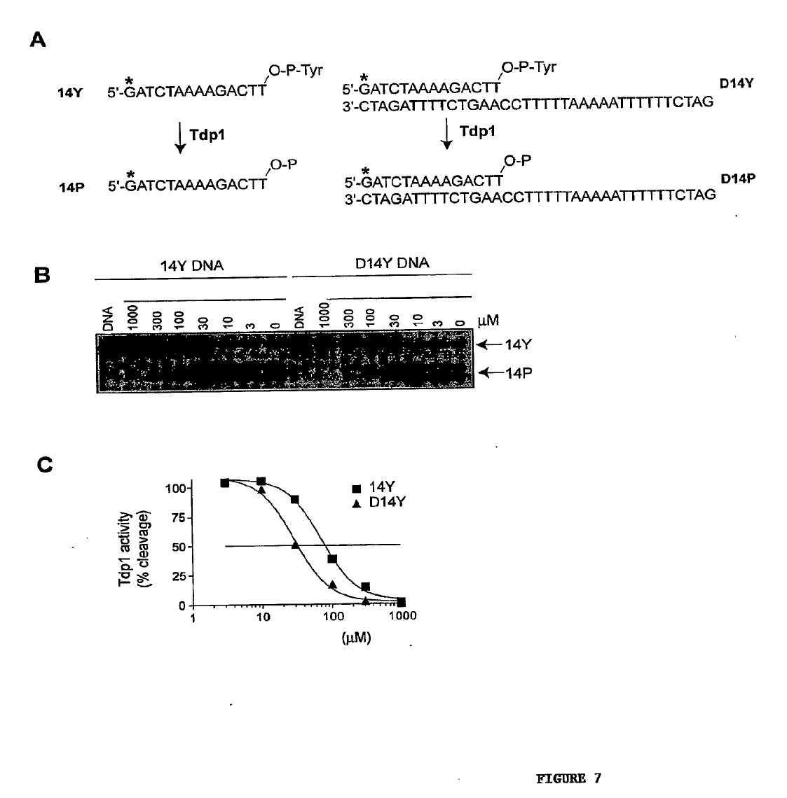

2,5-di-(4-phenylamidine)furan inhibits Tdp1 activity both with duplex and single-stranded substrates but is more effective with the duplex substrate. Having identified 2,5-di-(4-phenylamidine)furan as a novel Tdp1 inhibitor by the ECL assay, we evaluated the effect of 2,5-di-(4-phenylamidine)furan on Tdp1 activity in our gel-based assay (see FIG. 7). Since both partially duplex DNA (D14Y) and single-stranded DNA (14Y) are substrates for Tdp1 (Davies et al., J Mol Biol 324(5):917-932 (2002); Pouliot et al., Genes Cells 6(8):677-687 (2001); Yang et al., Proc Natl Acad Sci USA 93(21):11534-11539 (1996)), we compared the inhibition of Tdp1 by 2,5-di-(4-phenylamidine)furan using the D14Y and 14Y substrates (sequence as shown in FIG. 7A). As observed in FIGS. 7B and 7C, 2,5-di-(4-phenylamidine)furan inhibits the processing of both the single and double-stranded substrates by Tdp1 with an IC₅₀ of ˜30 and ˜90 mM, respectively.

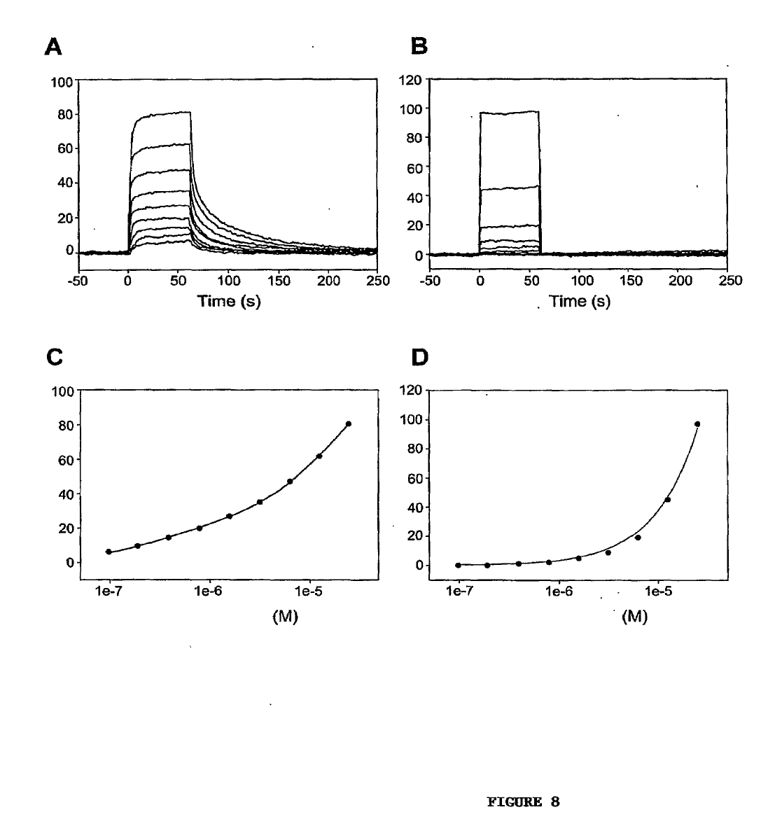

Preferential binding of 2,5-di-(4-phenylamidine)furan to a double-stranded substrate: The ability of 2,5-di-(4-phenylamidine)furan to directly interact with DNA was evaluated. Surface plasmon resonance analyses were carried out using single-stranded and double-stranded (stem-loop) substrates (for sequence, see materials and methods section above). As observed in FIG. 8A, 2,5-di-(4-phenylamidine)furan binds duplex oligonucleotide with a high affinity. 2,5-di-(4-phenylamidine)furan rapidly reaches a steady state binding level with duplex DNA but then disassociates more slowly. The equilibrium binding could only be fit using a 2 binding-site model with affinities of 0.33 and 19 mM (FIG. 8C). This seems reasonable given that the sequence AAGA that is contained within the oligonucleotide has previously been demonstrated to a high affinity binding site for a heterocyclic diamidine (Tanious et al., Biochemistry 42(46):13576-13586 (2003)). The high affinity binding 5 base-pair motif characterized by Tanious et al., has a capacity to form antiparallel dimers stacking with the DNA minor groove. Additionally, a duplex of 14 base-pairs could also support additional compound binding at lower affinity sites. However, with a single-stranded substrate, 2,5-di-(4-phenylamidine)furan both associates and disassociates very rapidly which most likely reflects the electrostatic interaction between the phosphate backbone and the charged compound (FIG. 8B). We estimate the Kd to be about 70 mM (FIG. 8D). We also evaluated the binding of compound to amine coupled Tdp1 protein (data not shown) and found the interaction to be very weak with a Kd of >900 mM. 2,5-di-(4-phenylamidine)furan does bind DNA with a preference for a duplex substrate.
Inhibition of Tdp1 by 2,5-di-(4-phenylamidine)furan is dependent on both reaction duration and Tdp1 concentration: A hallmark of all reversible inhibitors is that when the inhibitor concentration drops, enzyme activity is regenerated. Our initial gel assays (FIG. 7) were performed at a fixed time (20 min) under conditions where Tdp1 almost fully converts the substrate in the absence of inhibitor (1 ng, pH 8.0). We next evaluated the role of reaction time and enzyme concentration on the ability of 2,5-di-(4-phenylamidine)furan to inhibit Tdp1.

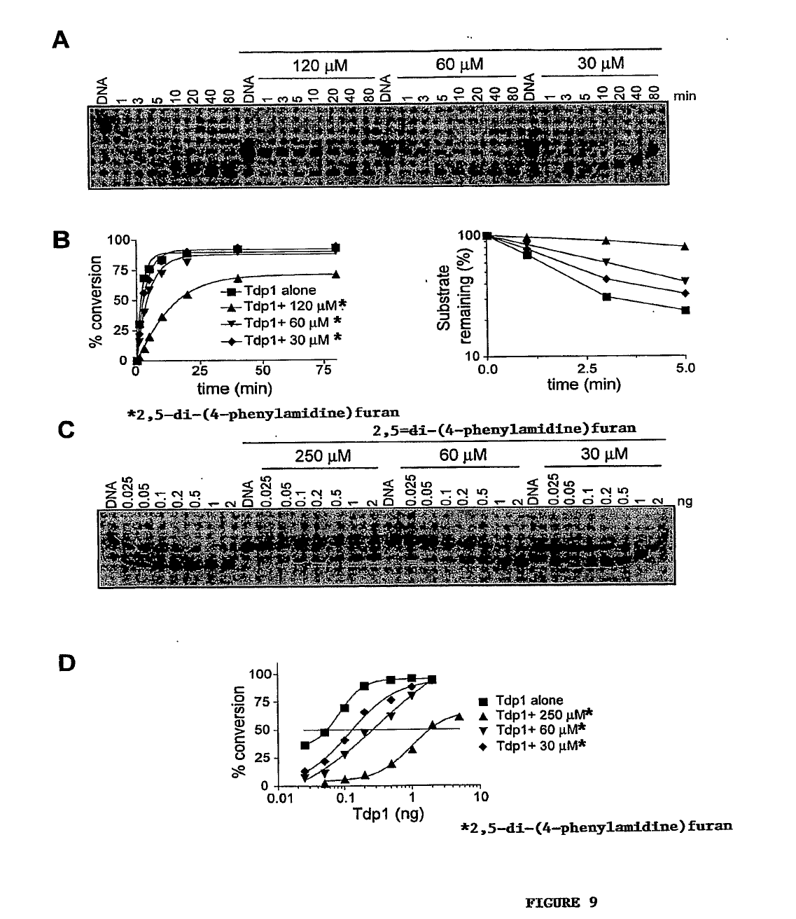

As shown in FIGS. 9A and 9B, (9A, left; and squares in 9B) 1 ng of Tdp1 converted about 50% (t) of the 14Y substrate within ˜1.9 min. Thus, we wished to determine whether concentrations of 2,5-di-(4-phenylamidine)furan below its determined IC₅₀ (˜90 μM) would affect the kinetics of Tdp1 activity. Tdp1 activity was slowed down even at 30 μM 2,5-di-(4-phenylamidine)furan (FIG. 9A). Kinetic plots demonstrated that 30 μM 2,5-di-(4-phenylamidine)furan increased the conversion half-time (t) of 14Y from 1.9 min in the absence of drug to 2.7 min in the presence of 30 μM 2,5-di-(4-phenylamidine)furan (diamond in FIG. 9B) and 4.4 min in the presence of 60 μM 2,5-di-(4-phenylamidine)furan (inverted triangle in FIG. 9B). Additionally, increasing Tdp1 concentration was able to overcome Tdp1 inhibition by 2,5-di-(4-phenylamidine)furan (FIGS. 9C and 9D). The 50% inhibition of Tdp1 activity observed by 30 μM 2,5-di-(4-phenylamidine)furan with 0.1 ng of Tdp1 was almost completely reversed by increasing the concentration of Tdp1 to 1 ng (FIG. 9C and diamond in FIG. 9D). Similar effect was seen with 60 μM and 250 μM 2,5-di-(4-phenylamidine)furan (FIGS. 9C and D). Thus, free Tdp1 competes with 2,5-di-(4-phenylamidine)furan. Moreover, if DNA were the only target of 2,5-di-(4-phenylamidine)furan, inhibition should not depend on Tdp1 concentration, which is not what we observed (FIGS. 5 C and D). These results indicate that 2,5-di-(4-phenylamidine)furan produces reversible inhibition of Tdp1.

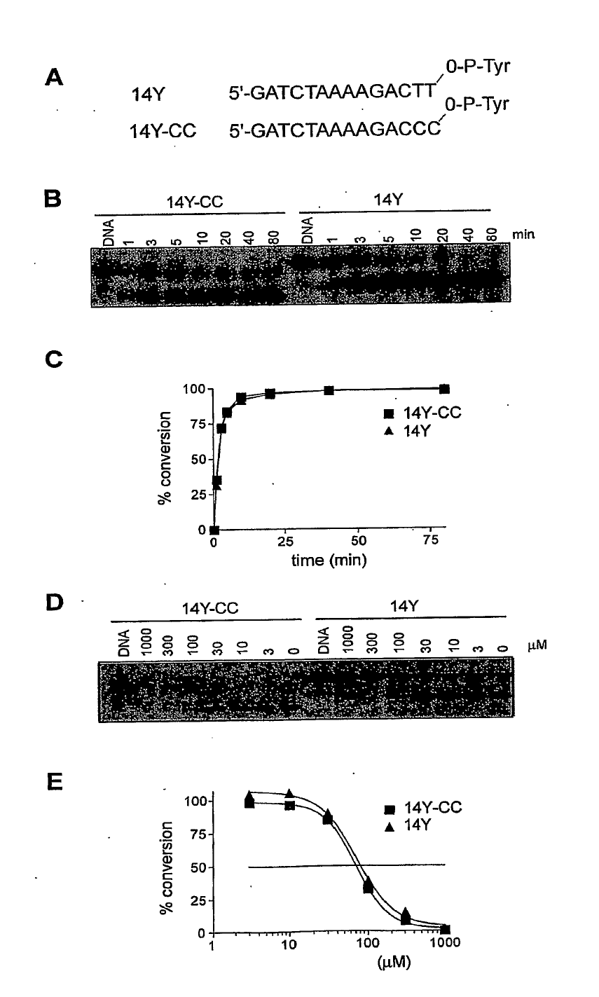

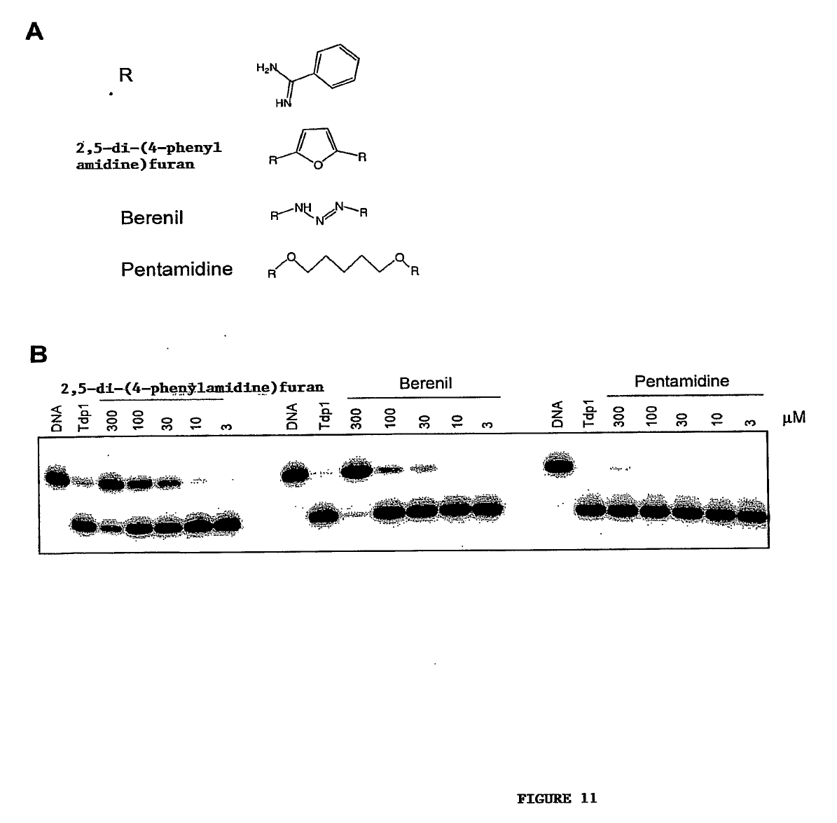

2,5-di-(4-phenylamidine)furan mediated inhibition of Tdp1 is Independent of the substrate sequence: The effect of altering the sequence of the substrate on the inhibition of Tdp1 by 2,5-di-(4-phenylamidine)furan was evaluated. The terminal thymine dinucleotide (-TT) of the 14Y oligonucleotide was replaced with a cytosine dinucleotide (-CC) to generate the 14Y-CC oligonucleotide (see FIG. 10A). No difference in the ability of Tdp1 to process either the 14Y or 14Y-CC substrates was observed (FIGS. 10B and 10C). Kinetic plot analysis shows both substrates processed almost completely within 10 min of the reaction time and at the same rate by 1 ng of Tdp1 (FIG. 10C). Upon addition of varying concentrations of 1,4-di-(4-phenylmidine)furan, the processing of both substrates 14Y and 14Y-CC by Tdp1 was inhibited to the same degree with an IC₅₀ of ˜90 μM (FIGS. 10D and 10E). Thus, substrate sequence at the termini is not critical for Tdp1 inhibition by the dication 2,5-di-(4-phenylamidine)furan.
2,5-di-(4-phenylamidine)furan inhibits Tdp1 more effectively than Berenil and Pentamidine: 2,5-di-(4-phenylamidine)furan, berenil and pentamidine were evaluated for their ability to inhibit Tdp1 activity in the 14Y substrate. FIG. 11B shows that pentamidine did not inhibit Tdp1 activity under these conditions. Berenil showed some activity, albeit at a high concentration (300 μM). 2,5-di-(4-phenylamidine)furan on the other hand, exhibits an inhibition of Tdp1 activity at 30 μM (FIG. 7B) and therefore is the most potent

### Incorporation by Reference

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. A method of inhibiting Tdp1 activity in a subject, the method comprising administering to the subject a diamidine compound.

2. The method of claim 1 wherein the diamidine compound is capable of modulating the activity of Tdp1.

3. The method of claim 1 wherein the diamidine compound comprise a furanyl moiety.

4. The method of claim 1, wherein the diamidine compound is a compound of Formula I: 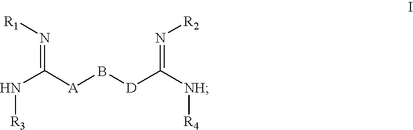
wherein,
A, B and D are each independently C₁-C₆ alkylene, C₃-C₁₀ cycloalkylene, C₁-C₉ heterocycloalkylene, C₆-C₁₀ arylene, C₁-C₁₀ heteroarylene, or absent;
R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted;
R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and
each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted;
or a pharmaceutically-acceptable salt thereof.

5. The method of claim 4 wherein A and D are each C₆-C₁₀arylene and B is heteroarylene.

6. The method of claim 4 wherein B is furanylene.

7. The method of claim 1 wherein the administered compound is of the following formula IA: 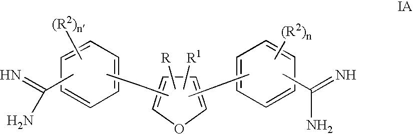
wherein R, R¹ and each R² are independently hydrogen or a non-hydrogen substituent; n and n′ are each independently integers from 0 to 4; and pharmaceutically acceptable salts thereof.

8. The method of claim 1 wherein the compound is:

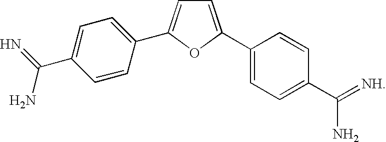

9. The method of claim 1 wherein the compound is:

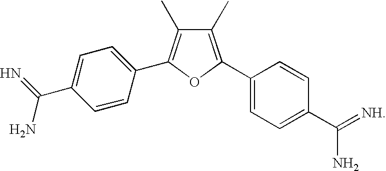

10. A method of treating a Tdp1-related disorder in a subject, comprising:
a) identifying a subject as being in need of a Tdp1 inhibitor;
b) administering to the subject in need thereof an effective amount of a diamidine compound.

11. The method of claim 10 wherein the Tdp1-related disorder is cancer, tumor, neoplasm, neovascularization, vascularization, cardiovascular disease, intravasation, extravasation, metastasis, arthritis, infection, Alzheimer's Disease, blood clot, atherosclerosis, melanoma, skin disorder, rheumatoid arthritis, diabetic retinopathy, macular edema, or macular degeneration, inflammatory and arthritic disease, or osteosarcoma.

12. The method of claim 10 wherein the diamidine compound comprises a furanyl moiety.

13. The method of claim 10 wherein the administered compound is of the following formula IA: 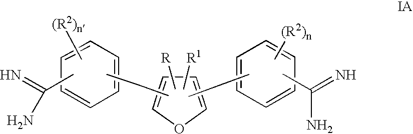
wherein R, R¹ and each R² are independently hydrogen or a non-hydrogen substituent; n and n′ are each independently integers from 0 to 4; and pharmaceutically acceptable salts thereof.

14. The method of claim 10 wherein the diamidine compound is a compound of Formula I: 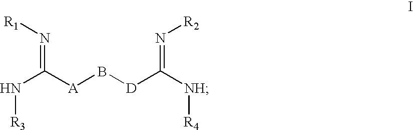
wherein,
A, B and D are each independently C₁-C₆ alkylene, C₃-C₁₀ cycloalkylene, C₁-C₉ heterocycloalkylene, C₆-C₁₀arylene, C₁-C₁₀ heteroarylene, or absent;
R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted;
R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and
each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted;
or a pharmaceutically-acceptable salt thereof.

15. The method of claim 14 wherein A and D are each C₆-C₁₀arylene and B is heteroarylene.

16. The method of claim 14 wherein B is furanylene.

17. The method of claim 10 wherein the administered compound is of the following formula IA: 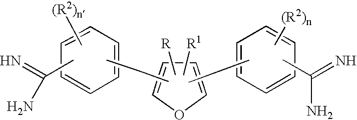
wherein R, R¹ and each R² are independently hydrogen or a non-hydrogen substituent; n and n′ are each independently integers from 0 to 4; and pharmaceutically acceptable salts thereof.

18-19. (canceled)

20. A method of treating cancer in a subject identified as in need of such treatment, the method comprising administering to said subject an effective amount of a compound of Formula I: 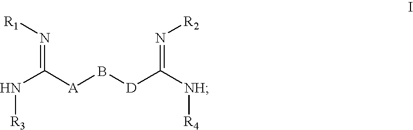
wherein,
A, B and D are each independently C₁-C₆ alkylene, C₃-C₁₀ cycloalkylene, C₁-C₉ heterocycloalkylene, C₆-C₁₀arylene, C₁-C₁₀heteroarylene, or absent;
R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted;
R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and
each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted;
or a pharmaceutically-acceptable salt thereof.

21. The method of claim 20 wherein A and D are each C₆-C₁₀ arylene and B is heteroarylene.

22. The method of claim 20 wherein B is furanylene.

23. The method of claim 20 wherein the compound is of the following formula IA: 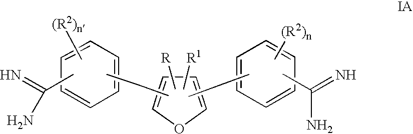
wherein R, R¹ and each R² are independently hydrogen or a non-hydrogen substituent; n and n′ are each independently integers from 0 to 4; and pharmaceutically acceptable salts thereof.

24. The method of claim 20 wherein the compound of Formula I is:

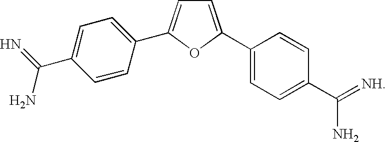

25. The method of claim 20 wherein the compound of Formula I is:

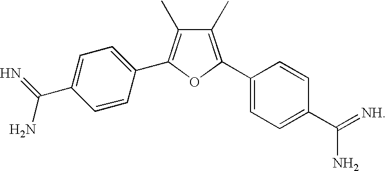

26. The method of claim 20 wherein the compound is a Tdp1 inhibitor.

27. The method of claim 20 further comprising an additional therapeutic agent.

28-30. (canceled)

31. The method of claim 1 wherein the subject is a human.

32. A pharmaceutical composition comprising a compound of Formula I: 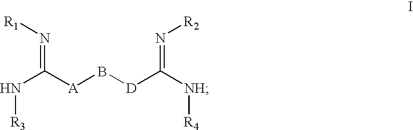
wherein,
A, B and D are each independently C₁-C₆ alkylene, C₃-C₁₀ cycloalkylene, C₁-C₉ heterocycloalkylene, C₆-C₁₀arylene, C₁-C₁₀ heteroarylene, or absent;
R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted;
R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and
each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted;
or a pharmaceutically-acceptable salt thereof;
together with a pharmaceutically-acceptable carrier or excipient.

33-37. (canceled)

38. A compound of Formula I: 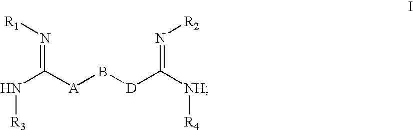
wherein,
A, B and D are each independently C₁-C₆ alkylene, C₃-C₁₀ cycloalkylene, C₁-C₉ heterocycloalkylene, C₆-C₁₀arylene, C₁-C₁₀ heteroarylene, or absent;
R₁-R₄ are each independently H, alkyl, cycloalkyl, heterocycloalkyl, aryl, heteroaryl, aralkyl, heteroaralkyl, hydroxylalkyl, —C(O)R^{a}, —C(S)R^{a}, —C(NR)R^{a}, haloalkyl, —S(O)R^{a}, —S(O)₂R^{a}, —P(O)R^{a}R^{a}, —P(S)R^{a}R^{a}, or alkylcarbonylalkyl; each of which may be optionally substituted;
R^{a} is independently for each occurrence H, alkyl, alkenyl, alkynyl, cycloalkyl, heterocycloalkyl, aralkyl, heteroaralkyl, aryl, heteroaryl, haloalkyl, —OR^{b}, —SR^{b}, —NR^{b}R^{b}, hydroxylalkyl, alkylcarbonylalkyl, mercaptoalkyl, aminoalkyl, sulfonylalkyl, sulfonylaryl, or thioalkoxy; each of which may be optionally substituted; and wherein two or more R^{a} groups, when attached to a heteroatom, may together form a heterocyclic ring with said heteroatom, wherein the heterocyclic ring may be optionally substituted; and
each R^{b} is independently H, alkyl, alkenyl, alkynyl, cycloalkyl, heterocyclyl, heterocycloalkyl, aralkyl, heteroaralkyl, aryl, or heteroaryl; each of which may be optionally substituted;
or a pharmaceutically-acceptable salt thereof.

39. A compound of claim 38 wherein A and D are each C₆-C₁₀ arylene and B is heteroarylene.

40-53. (canceled)


SEQUENCE LISTING (5 sequences)

SEQ ID NO: 1 - protein, 25 aa, Artificial Sequence
  source: /note=“Description of Artificial Sequence Synthetic peptide”
MGSSHHHHHHSSGLVPRGSHMLEDP

SEQ ID NO: 2 - DNA, 32 bp, Artificial Sequence
  source: /note=“Description of Artificial Sequence Synthetic oligonucleotide”
gatctaaaagactttctcaagtcttttagatc

SEQ ID NO: 3 - DNA, 14 bp, Artificial Sequence
  source: /note=“Description of Artificial Sequence Synthetic oligonucleotide”
gatctaaaagactt

SEQ ID NO: 4 - DNA, 36 bp, Artificial Sequence
  source: /note=“Description of Artificial Sequence Synthetic oligonucleotide”
gatcttttttaaaaatttttccaagtcttttagatc

SEQ ID NO: 5 - DNA, 14 bp, Artificial Sequence
  source: /note=“Description of Artificial Sequence Synthetic oligonucleotide”
gatctaaaagaccc